(19) United States
(12) Patent Application Publication
(10) Pub. No.: US 2002/0138123 A1
(43) Pub. Date: Sep. 26, 2002

(54) Medical electrical leads and indwelling catheters with enhanced biocompatibility and biostability

(75) Inventors: Jesus W. Casas-Bejar, Brooklyn Park, MN (US); Darrel F. Untereker, Cedar, MN (US); Maura G. Donovan, St. Paul, MN (US); Zhao Qinghong, Andover, MN (US); Brian C.A. Fernandes, Roseville, MN (US); Timothy H. Robinson, Savage, MN (US); Peter T. Schroeder, Minneapolis, MN (US)

Correspondence Address: Kenneth J. Collier, Medtronic, Inc., 710 Medtronic Parkway, Minneapolis, MN, 55432-5604, US

(73) Assignee: Medtronic, Inc.

(21) Appl. No.: 09/998,536

(22) Filed: Nov. 28, 2001

Related U.S. Application Data
(63) Continuation of application No. 09/063,277, filed on Apr. 21, 1998, now Pat. No. 6,154,192.

(51) Int. Cl. A61N 1/05

(52) U.S. Cl. 607/120

(57) ABSTRACT

A medical electrical lead or indwelling catheter comprising an elongated body having a tissue-contacting surface that includes a polymer in intimate contact with a steroidal anti-inflammatory agent.

### FIELD OF THE INVENTION

This invention relates generally to medical electrical leads and indwelling catheters with enhanced biocompatibility and biostability. More particularly, the present invention relates to medical electrical leads and indwelling catheters having a body portion comprising a polymer in intimate contact with a steroidal anti-inflammatory agent.

### BACKGROUND OF THE INVENTION

The use of implants and medical devices has become widely accepted in the various clinical fields, and has shown a tremendous growth during the past three decades. Clinical use of these mostly synthetic devices is not completely free of complications, however. For example, device-associated infections can require implant removal. Degradation of the polymeric components of implants can also necessitate implant removal. This is even true of biocompatible polymers such as polyurethanes (specifically, polyetherurethanes). Even such biocompatible polymers can trigger the body's defensive mechanisms in response to foreign materials, which can eventually cause stress cracking, for example.

Implant removal, however, can be detrimental to the surrounding tissue, particularly if the tissue has encased or encapsulated the implant. For example, cardiac tissue can surround the body of a medical electrical lead to such an extent that when removal is necessary a portion of the lead body may need to remain in place to avoid damaging the surrounding tissue (e.g., cardiac tear or rupture) and even death. Such encapsulation can result from the long-term chain of events involved in the wound-healing response, which is initially characterized by acute and chronic inflammation.

Extensive polymer research is being done, particularly in the area of chemical modifications of materials, to develop materials that are resistant to biodegradation. Although materials with increased resistance to hydrolysis and oxidation can be formulated, certain modifications may effect their biocompatibility. To prevent encapsulation, a few approaches have been tried that have focused on the use of antithrombogenic and “bioactive” surfaces. None of these approaches, however, have been effective at controlling the long term sequence of events that takes place at the interface between bodily tissues and biomaterials and results in the formation of encapsulating tissue.

Medical devices containing polymers are known to include therapeutic agents for delivery to surrounding tissue. For example, stents have been designed with polymeric coatings or films that incorporate a wide variety of therapeutic agents, such as anti-inflammatory agents, anti-thrombogenic agents, and anti-proliferative agents, for a wide variety of purposes. Antimicrobial compounds have been incorporated into polymeric portions of medical devices for sustained release to the surrounding tissue to enhance infection-resistance. Medical electrical leads have incorporated steroids into or at the lead tip electrode, to reduce source impedance and lower peak and chronic pacing thresholds. However, to date anti-inflammatory agents have not been recognized as useful for effecting the biocompatibility and/or biostability of biomaterials used in implantable medical devices, particularly those that may need to be removed.

Many of the following lists of patents and nonpatent documents disclose information related to medical devices (e.g., stents and lead tips) containing anti-inflammatory agents, particularly steroids. Others in the following lists relate to biomaterials and human response mechanisms.

TABLE 1a
Patents

| Patent No. | Inventor(s) | Issue Date |
|---|---|---|
| 4,506,680 | Stokes | Mar 26 1985 |
| 4,577,642 | Stokes | Mar 25 1986 |
| 4,585,652 | Miller et al. | Apr 29 1986 |
| 4,784,161 | Skalsky et al. | Nov 15 1988 |
| 4,873,308 | Coury et al. | Oct 10 1989 |
| 4,972,848 | Di Domenico et al. | Nov. 27 1990 |
| 4,922,926 | Hirschberg et al. | May 8 1990 |
| 5,002,067 | Berthelsen et al. | Mar 26 1991 |
| 5,009,229 | Grandjean et al. | Apr 23 1991 |
| 5,092,332 | Lee et al. | Mar 3 1992 |
| 5,103,837 | Weidlich et al. | Apr 14 1992 |
| 5,229,172 | Cahalan et al. | Jul 20 1993 |
| 5,265,608 | Lee et al. | Nov 30 1993 |
| 5,282,844 | Stokes et al. | Feb 1 1994 |
| 5,324,324 | Vachon et al. | Jun 28 1994 |
| 5,344,438 | Testerman et al. | Sep 6 1994 |
| 5,408,744 | Gates | Apr 25 1995 |
| 5,431,681 | Helland | Jul 11 1995 |
| 5,447,533 | Vachon et al. | Sep 5 1995 |
| 5,510,077 | Dinh et al. | Apr 23 1996 |
| 5,554,182 | Dinh et al. | Sep 10 1996 |
| 5,591,227 | Dinh et al. | Jan 7 1997 |
| 5,599,352 | Dinh et al. | Feb 4 1997 |
| 5,609,629 | Fearnot et al. | Mar 11 1997 |
| 5,679,400 | Tuch | Oct 21 1997 |
| 5,624,411 | Tuch | Apr 29 1997 |
| 5,727,555 | Chait | Mar 17 1998 |

TABLE 1b
Nonpatent Documents

Ackerman et al., “Purification of Human Monocytes on Microexudate-
Coated Surfaces,” J. Immunol., 20, 1372-1374 (1978)
Alderson et al., “A Simple Method of Lymphocyte Purification from
Human Peripheral Blood,” J. Immunol. Methods, 11, 297-301 (1976)
Anderson, “Mechanisms of Inflammation and Infection with Implanted
Devices,” Cardiovasc. Pathol., 2, 335-415 (1993)
Anderson, “Inflammatory Response in Implants,” ASAIO, 11, 101 (1988)
Boyum et al., “Density-Dependent Separation of White Blood Cells,”
Blood Separation and Plasma Fractionation, 217-239, Wiley-Liss, Inc.
(1991)
Bonfield et al., “Cytokine and Growth Factor Production by
Monocytes/Macrophages on Protein Preadsorbed Polymers,” J. Biom.
Mat. Res., 26, 837-850 (1992)
Cardona et al., “TNF and IL-1 Generation by Human Monocytes in
Response to Biomaterials,” J. Biom. Mat. Res., 26, 851-859 (1992)
Casas-Bejar et al., “In vitro Macrophage-Mediated Oxidation and Stress
Cracking in a Polyetherurethane,” Transactions of the Fifth World
Biomaterials Cogress, Toronto, Canada (1996)
Fujimoto et al., “Ozone-Induced Graft Polymerization onto Polymer
Surface,” J. Polym. Chem., 31, 1035-1043 (1993)
Kao et al., “Rote of Interleukin-4 in Foreign Body Giant Cell Formation
on a Poly(etherurethane urea) in vivo,” J. Biomed. Mat. Res., 29,
1267-1275 (1995)
Merchant et al., “In vivo Biocompatibitity Studies. I. The Cage Implant
System and a Biodegradable Hydrogel,” J. Biomed. Mat. Res., 17, 301-
325 (1983)
Miller et al., “Human Monocyte/Macrophage Activation and Interleukin-1
Generation by Biomedical Polymers,” J. Biom. Mat. Res., 22, 713-731
(1988)
Mond et al., “The Steroid-Eluting Electrode: A 10-Year Experience,”
PACE, 19, 1016-1020 (1996)
Schubert et al., “Oxidative Biodegradation Mechanisms of Biaxially
Strained Poly(etherurethane urea) Elastomers,” J. Biomed. Mat. Res.,
29, 337-347 (1995)
Shanbhag et al., “Macrophage/Particle Interactions: Effects of Size,
Composition and Surface Area,” J. Biomed. Mater. Res., 28, 81-90
(1994)
Stokes et al., “Polyurethane Elastomer Biostability,” J. of Biomaterials
Applications, 9, 321-354 (1995)
Zhao et al., “Cellular Interactions and Biomaterials: In vivo Cracking of
Pre-stressed Pellethane 2363-80A,” J. Biom. Mat. Res., 24, 621-637
(1990)
Zhao et al., “Glass Wool-H₂O₂/CoCl₂ Test System for the In Vitro
Evaluation of Biodegradative Stress Cracking in Polyurethane
Elastomers,” J. Biomed. Mat. Res., 29, 467-475 (1995)

All patent and nonpatent documents listed in Table 1 are hereby incorporated by reference herein in their respective entireties. As those of ordinary skill in the art will appreciate upon reading the Summary of the Invention, Detailed Description of Preferred Embodiments, and claims set forth below, many of the devices and methods disclosed in these documents may be modified advantageously by using the teachings of the present invention.

### SUMMARY OF THE INVENTION

The present invention is directed at enhancing the biocompatibility and/or biostability of polymers in implantable medical devices. To do this, the present invention does not involve modifying the chemistries of the polymers, rather it involves using anti-inflammatory agents as biological response modulators to “protect” the polymers.

As used herein, the term “biostable” refers to an organic polymer's chemical and physical stability during implantation in living tissue. More specifically, it refers to resistance to the degradative phenomena to which the polymer is exposed during the acute and chronic host response (e.g., inflammation). In the context of the present invention, improving the biostability of a polymer does not involve changing the chemistry of the polymer; rather, it focuses on down-regulating the cellular attack. Thus, as used herein, biostability refers to the effects of cells and tissues on materials.

As used herein, the term “biocompatible” refers to the degree of host response elicited by an organic polymer upon implantation. Typically, this is evaluated by assessing the inflammatory phenomenon, particularly in surrounding tissues. Less inflammation or biological disturbance suggests better biocompatibility and vice versa. Thus, as used herein, biocompatibility refers to the effects of materials on cells and tissues.

Thus, various embodiments of the present invention are intended to fulfill one or more of the following objects: to enhance material biocompatibility; to enhance material biostability; to reduce acute inflammation; to reduce chronic inflammation; and to reduce fibrous tissue formation (e.g., reduced tissue encapsulation).

In one embodiment, the present invention provides a medical electrical lead comprising: an elongated insulative lead body having a tissue-contacting surface, a proximal end, and a distal end; an elongated conductor having a proximal end and a distal end, mounted within the insulative lead body; an electrode coupled to the distal end of the electrical conductor for making electrical contact with bodily tissue; wherein the tissue-contacting surface of the insulative lead body comprises a polymer in intimate contact with a steroidal anti-inflammatory agent, preferably, a glucocorticosteroid, such as dexamethasone, a derivative thereof, or a salt thereof. The anti-inflammatory agent can be coated onto, or impregnated into, or covalently bonded to, the tissue-contacting surface, for example. Preferably, the tissue-contacting surface consists essentially of a nonporous polymer in intimate contact with a steroidal anti-inflammatory agent.

In another embodiment, the present invention provides an indwelling catheter comprising: an elongate body having a proximal end, a distal end, a tissue-contacting surface, and at least one interior lumen therethrough; and an external fitting coupled to the proximal end; wherein the tissue-contacting surface of the elongate body comprises a polymer in intimate contact with a steroidal anti-inflammatory agent, preferably, a glucocorticosteroid, such as dexamethasone, a derivative thereof, or a salt thereof. The anti-inflammatory agent can be coated onto, or impregnated into, or covalently bonded to, the tissue-contacting surface, for example. Preferably, the tissue-contacting surface consists essentially of a nonporous polymer in intimate contact with a steroidal anti-inflammatory agent. The indwelling catheter also preferably includes one or more helical coils formed in the elongate body between the proximal and distal ends.

As used herein, the term “proximal” means that portion of a lead or indwelling catheter which is disposed in closer proximity to the end of the lead or catheter that remains outside a patient's body during an implantation procedure than to the end of the lead or catheter that is inserted first inside the patient's body during an implantation procedure.

The term “distal” means that portion of a lead or indwelling catheter which is disposed in closer proximity to the end of the lead or catheter that is inserted first into a patient's body during an implantation procedure than to the end of the lead or catheter that remains outside the patient's body during an implantation procedure.

Significantly, these devices can be used to modulate tissue encapsulation and polymer degradation when implanted into a patient. Thus, the present invention also provides methods of modulating tissue encapsulation or degradation of a medical electrical lead or indwelling catheter by implanting the leads and catheters described above. The present invention also provides a variety of methods for making the medical electrical leads and indwelling catheters described above.

### BRIEF DESCRIPTION OF THE FIGURES

FIG. 1 is a side plan view of one embodiment of a medical electrical lead according to the present invention.

FIG. 2 is a schematic of an implantable device having medical electrical leads according to the present invention shown in the body of a patient.

FIG. 3 is a side plan view of one embodiment of an indwelling catheter according to the present invention.

FIG. 4 is a graph showing in vitro hydroperoxide formation in: standard culture media (no cells) containing polyetherurethane specimens (presoaked in acetone “AS”); polyetherurethane (AS) specimens stored in the dark under ambient conditions; and standard culture media with rabbit Mo/MØs containing polyetherurethane (AS) specimens.

FIG. 5 is a graph showing in vitro hydroperoxide formation in: standard culture media with human Mo/MØs containing polyetherurethane specimens (with and without presoaking in acetone); standard culture media with human lymphocytes containing polyetherurethane (AS) specimens; and standard culture media with human Mo/MØs containing polyetherurethane (AS) specimens plus dexamethasone sodium phosphate at 0.024 μg/ml (+) and 240 μg/ml (+++)

FIG. 6 is a bar chart of hydroperoxide concentration in polymer specimens with and without dexamethasone after a 40-day macrophage treatment step.

FIG. 7 shows a graph of the amount of dexamethasone elution per material surface area (cm²) over a period of 32 days.

FIG. 8 is a bar chart showing graphically the overall environmental stress cracking in explants at 6 weeks and 10 weeks. Data were summarized using the highest (most severe) score of surface damage observed in the explanted biostability samples. Optical microscopic observation at 70× total magnification.

FIG. 9 is a graphical representation of the comparative total cell count in cage exudate in response to different PU materials: 1D=1% dexamethasone in polyurethane; 20D=20% dexamethasone in polyurethane; A=control; and EC=empty cage.

FIG. 10 is graphical representation of in vitro elution of dexamethasone from dexamethasone-coated leads. “Low” (1%DEX/PU) and “High” (5% DEX/PU) loadings were used. Elution percentages of the total theoretical dexamethasone loading was determined in PBS at 37° C.

FIG. 11 is graphical representation of in vitro elution of dexamethasone from dexamethasone-coated leads per surface area vs. time. “Low” (1%DEX/PU) and “High” (5% DEX/PU) loadings were used. Elution was conducted in PBS at 37° C.

FIG. 12 is graphical representation of in vitro elution of dexamethasone from dexamethasone-coated leads following 90 days in vivo implantation. “Low” (1%DEX/PU) and “High” (5% DEX/PU) loadings were used. Elution percentages of the total theoretical dexamethasone loading was determined in PBS at 37° C.

### DETAILED DESCRIPTION OF THE INVENTION

The present invention is directed at enhancing the biocompatibility and/or biostability of polymeric materials by modulating cellular behavior involved in biological defensive mechanisms, such as phagocytosis and enzymatic and oxidative mechanisms. This does not involve modifying the chemistries of the polymers per se, rather it involves using biological response modulators to “protect” the polymers. Significantly, it has been discovered that elution of such biological response modulators at the interface between the polymer and the surrounding tissue (solid or liquid tissues, e.g., blood), modulates the behavior of cells at that interface. As a result, the polymer is exposed to fewer cell-produced damaging agents, such as reactive oxygen species. In essence, the defensive mechanisms of cells in response to foreign materials is down-regulated by the present invention.

Thus, the present invention provides an implantable medical device (i.e., implant) having a tissue-contacting surface that includes a polymer in intimate contact (i.e., direct contact) with an anti-inflammatory agent for modulating the behavior of cells in contact with the tissue-contacting surface. Significantly, the anti-inflammatory agent moderates certain cellular activities at the site of the implant that causes inflammation, for example. Such cellular activity includes exuberant tissue growth and oxidative burst. Exuberant tissue growth refers to fibrous tissue formation as a result of cellular proliferation and deposition of extracellular components, including collagen, elastin, and fibronectin. It tends to cause encapsulation of the implant, which can be detrimental particularly when it becomes desirable to remove the implant. Oxidative burst refers to the ability of phagocytes to consume oxygen and produce reactive oxygen species such as hydroxyl radicals, superoxide, hydrogen peroxide, and other reactive oxides and peroxides. It tends to cause degradation of the polymer of which the implant is made.

The anti-inflammatory agent is preferably localized at the tissue-contacting surface of the medical device. Alternatively, it can be eluted from a remote site within the medical device, as long as upon elution it is in intimate contact with the polymer at the tissue-contacting surface of the medical device. Although the inventors do not wish to be bound by theory, it is believed that the anti-inflammatory agent is then released from the medical device. Initial release of the anti-inflammatory agent at the site of implantation is believed to reduce cell-associated propagation of the inflammatory signal. Sustained release is believed to maintain a low level of activation and differentiation of cells that come in contact with the tissue-contacting surface.

The present invention provides one or-more of the following desirable effects: enhanced material biocompatibility; enhanced material biostability; reduced acute inflammation; reduced chronic inflammation; and reduced fibrous tissue formation (e.g., reduced tissue encapsulation).

Anti-Inflammatory Agents

Suitable anti-inflammatory agents for use in the present invention are steroids. Preferably, the steroids are glucocorticoids, salts, and derivatives thereof. Examples of such steroids include cortisol, cortisone, fludrocortisone, Prednisone, Prednisolone, 6α-methylprednisolone, triamcinolone, betamethasone, dexamethasone, beclomethasone, aclomethasone, amcinonide, clebethasol, clocortolone. Dexamethasone (9α-fluoro-11β,17α,21-trihydroxy-16α-methylpregna-1,4-diene-3,20-ione), derivatives thereof, and salts thereof are particularly preferred. Dexamethasone sodium phosphate and dexamethasone acetate are suitable salts and dexamethasone-21-orthophosphate and its disodium salt are suitable derivatives.

The anti-inflammatory agent can be used in any amount that produces the desired response without detrimental effects, such as cytotoxic effects or the suppression of the immune response. Typically, it is used in an amount or dosage appropriate for the desired duration and intensity of the anti-inflammatory effect. Ultimately, this is dictated by the type of device to which this invention is applied. Generally, it is believed, however, that less than about 1 mg of an anti-inflammatory agent per square centimeter of surface area of a polymer-contacting surface can be used to produce the advantageous results described herein.

Tissue-Contacting Surface

The organic polymer of the tissue-contacting surface of the implantable medical device can be in the form of a tube, sheath, sleeve, coating, or the like. Typically, for the embodiments described herein, the polymer is in the form of a tube or sheath. It can be extruded, molded, coated on another material (e.g., metal), grafted onto another material, embedded within another material, adsorbed to another material, etc. The choice of polymer includes those that are not intended for tissue in-growth. Typically, such polymers are solid (i.e., nonporous) and are intended to be in contact with bodily tissues for extended periods of time (e.g., days, months, years). They are used in long-term implants such as medical electrical leads and indwelling catheters.

Although the polymers of the tissue-contacting surface are nonporous, this does not mean that the therapeutic agent cannot migrate out of the polymer if it is incorporated therein; rather, this means that the tissue-contacting surface does not include a porous material as is known in the art, such as that disclosed in U.S. Pat. No. 5,609,629 (Fearnot et al.) and U.S. Pat. No. 5,591,227 (Dinh et al.).

Examples of such polymers include a polyurethane, such as a polyether urethane, or any of the well known biostable polymeric materials typically used in implantable devices. These include, but are not limited to: silicones; polyamides, such as nylon-66; polyimides; polycarbonates; polyethers; polyesters, such as polyethylene terephthalate; polyvinyl aromatics, such as polystyrenes; polytetrafluoroethylenes; polyolefins, such as polyethylenes, polypropylenes, polyisoprenes, and ethylene-alpha olefin copolymers; acrylic polymers and copolymers; vinyl halide polymers and copolymers, such as polyvinyl choride; polyvinyl ethers, such as polyvinyl methyl ether; polyvinyl esters, such as polyvinyl acetate; polyvinyl ketones; polyvinylidine halides, such as polyvinylidene fluoride and polyvinylidene chloride; polyacrylonitrile; as well as copolymers of vinyl monomers with each other and olefins, such as ethylene-methyl methacrylate copolymers, acrylonitrile-styrene copolymers, ABS resins, and ethylene-vinyl acetate copolymers. Polyurethanes and silicones, or combinations thereof, are presently the preferred polymeric substrates in the context of this invention.

Surface Treatment

The tissue-contacting surface includes a polymer as described above in intimate contact with an anti-inflammatory agent. The anti-inflammatory agent can be incorporated into the medical device in a variety of ways. For example, the anti-inflammatory agent can be covalently grafted to the polymer of the tissue-contacting surface, either alone or with a surface graft polymer. Alternatively, it can be coated onto the surface of the polymer either alone or intermixed with an overcoating polymer. It can be physically blended with the polymer of the tissue-contacting surface as in a solid-solid solution. It can be impregnated into the polymer by swelling the polymer in a solution of the appropriate solvent. Any means by which the anti-inflammatory agent can be incorporated into the medical device such that it is in intimate contact with the tissue-contacting surface of the device are within the scope of the present invention.

In one embodiment, the polymer of the tissue-contacting surface and an anti-inflammatory agent are intimately mixed either by blending or using a solvent in which they are both soluble (e.g., xylene for silicone and dexamethasone phospate). This mixture can then be formed into the desired shape and incorporated into the medical device or coated onto an underlying structure of the medical device.

Alternatively, an overcoating polymer, which may or may not be the same polymer that forms the primary polymer of the tissue-contacting surface, and an anti-inflammatory agent are intimately mixed, either by blending or using a solvent in which they are both soluble, and coated onto the tissue-contacting surface. The overcoating polymers are preferably any of the biostable polymers listed above, as long as they are able to bond (either chemically or physically) to the polymer of the tissue-contacting surface. Alternatively, however, they can be any of a wide variety of bioabsorbable polymers, as long as they are able to bond (either chemically or physically) to the polymer of the tissue-contacting surface. Examples of suitable bioabsorbable polymers include poly(L-lactic acid), polycaprolactone, poly(lactide-co-glycolide), poly(hydroxybutyrate), poly(hydroxybutyrate-co-valerate), and others as disclosed in U.S. Pat. No. 5,679,400 (Tuch).

Yet another embodiment includes swelling the polymer of the tissue-contacting surface with an appropriate solvent and allowing the anti-inflammatory agent to impregnate the polymer. For example, for polyurethane, tetrahydrofuran, N-methyl-2-pyrrolidone, and/or chloroform can be used.

In another embodiment, anti-inflammatory agent is covalently grafted onto the polymer of the tissue-contacting surface. This can be done with or without a surface graft polymer. Surface grafting can be initiated by corona discharge, UV irradiation, and ionizing radiation. Alternatively, the ceric ion method, previously disclosed in U.S. Pat. No. 5,229,172 (Cahalan et al.), can be used to initiate surface grafting.

Herein, whether an overcoating polymer or surface graft polymer are used, the tissue-contacting surface is defined to include this secondary polymer (e.g., overcoating polymer or surface graft polymer), as well as the primary polymer that forms the structure of the medical device (e.g., lead or catheter bodies). Such polymers are solid polymers i.e., nonporous). Thus, in the constructions of the present invention there is no coating of a porous material over the tissue-contacting surface with which the anti-inflammatory agent is in intimate contact.

Heparin, or similar therapeutic agents, however, can be incorporated into the medical device. Preferably, the heparin is also in contact with the tissue-contacting surface in an amount effective to prevent or limit thrombosis. Heparin can be incorporated by coating, covalently bonding, or any of a variety of well-known techniques for incorporating heparin into a medical device. In one embodiment, heparin is covalently bonded to the tissue-contacting surface containing an anti-inflammatory agent.

Medical Electrical Leads

In one embodiment of the present invention, the implantable medical device is an implantable medical electrical lead for the delivery of an electrical stimulus to a desired body site. In this embodiment, an anti-inflammatory agent for modulating the behavior of cells is in intimate contact with the tissue-contacting surface of the elongated body portion of the lead. Such medical electrical leads include those used in cardiac pacing and defibrillation (including unipolar or bipolar, atrial or ventricular, transvenous or epimyocardial, endocardial or epicardial), as well as other electrode technologies, including neurological and muscle stimulation applications.

FIG. 1 illustrates a plan view of an exemplary medical electrical lead in accordance with the present invention. The lead includes an elongated lead body 10 covered by an insulative sheath 12 (herein, referred to as an elongated insulative lead body). The insulative sheath 12 defines the tissue-contacting surface of the elongated lead body 10. This insulative sheath 12 includes a polymer in intimate contact with an anti-inflammatory agent. Preferably, the anti-inflammatory agent is in intimate contact with the polymer along a substantial portion of the length of the insulative sheath, although this is not a necessary requirement. The portion of the lead body that is in contact with the anti-inflammatory agent depends on the tissue and anatomical structure in which the lead will be implanted. If desired, the entire structure of a medical electrical lead can be coated with an anti-inflammatory agent.

The polymer of this tissue-contacting surface of a medical electrical lead is typically fabricated of a flexible biostable polymeric insulator, such as polyurethane, silicone rubber, combinations thereof, or other polymers as described above. Mounted within this elongated insulative lead body is an elongated conductor (not shown) having a proximal end and a distal end.

At the proximal end of the elongated lead body 10, terminal assembly 14 is adapted to couple the lead to an implantable pacemaker pulse generator. Terminal assembly 14 is provided with sealing rings 16 and a terminal pin 18, all of a type known in the art. An anchoring sheath 20 (shown partially in cross-section) slides over lead body 10 and serves as a point for suturing the lead body to body tissue at the insertion point of the lead into the vein or tissue in a fashion known in the art. Anchoring sheath 20 and terminal assembly 14 may be conveniently fabricated of silicone rubber, for example.

At the distal end of the elongated lead body, a tip electrode 22 is coupled to the electrical conductor for making electrical contact with bodily tissue (e.g., heart tissue). As shown in FIG. 1, a tine protector 15 is shown (in cross-section) protecting the tines until the lead is used. Tines 26 are employed to passively retain the tip electrode 22 in position as is well known in the pacing art. The tip electrode 22 shown in FIG. 1, is a ball-tip electrode, although other shapes are possible, including cylindrical, corkscrew, ring tip, and open cage configurations.

Implantable medical electrical leads of the present invention can also include a steroid eluting porous pacing electrode, as disclosed in U.S. Pat. No. 4,506,680 (Stokes) and U.S. Pat. No. 4,577,642 (Stokes), for example. Such porous electrodes can be constructed of sintered platinum, titanium, carbon, or ceramic compositions. Within the electrode, there can be a plug of a polymer (e.g., silicone rubber) impregnated with an elutable steroid. Such porous steroid eluting electrodes present a source impedance substantially lower compared to similarly sized solid electrodes and present significantly lower peak and chronic pacing thresholds than similarly sized solid or porous electrodes.

As shown in FIG. 2, implantable medical electrical leads 54 can be implanted into the heart 56 of a patient 50 and used with a variety of implantable medical devices, particularly pacing and/or defibrillating devices, such as a pacemaker/cardioverter/defibrillator (PCD) 52.

A medical electrical lead according to-the present invention can be made by a variety of methods. In one embodiment, a method includes: providing an elongated insulative lead body having a tissue-contacting surface, a proximal end, and a distal end; wherein the tissue-contacting surface comprises a polymer in intimate contact with a steroidal anti-inflammatory agent; providing an elongated conductor having a proximal end and a distal end; mounting the elongated conductor within the insulative lead body; and coupling an electrode to the distal end of the electrical conductor for making electrical contact with bodily tissue. Preferably, the step of providing an elongated insulative lead body comprises blending a steroidal anti-inflammatory agent with a polymer and forming a tissue-contacting surface. Alternatively, the step of providing an elongated insulative lead body comprises coating a steroidal anti-inflammatory agent onto the tissue-contacting surface of the lead body.

Indwelling Catheters

In one embodiment of the present invention, the implantable medical device is an indwelling catheter for use in applications where connection from the outside of the patient's body to an internal cavity within the body is desired, such as in the the gastrointestinal tract, biliary tree, the liver, the kidney, etc. In this embodiment, an anti-inflammatory agent for modulating the behavior of cells is in intimate contact with the tissue-contacting surface of the elongated body portion of the catheter. Such indwelling catheters include those used in the areas of gastrostomy, gastrojejunostomy, cecostomy, and the like. They can be used for chemotherapeutic drugs, feeding, etc.

With reference to gastrostomy and gastrojejunostomy procedures as a particular example, catheters for use in these procedures are inserted directly through the abdominal wall of the patient and into the stomach. Gastrostomy catheters can then be used for feeding the patient directly into the stomach, wherein nourishing substances are inserted into an external opening in the catheter and are transported by the catheter to the interior of the patient's stomach. With the gastrojejunostomy catheter, the distal portion of the catheter inside the patient is long enough to be positioned in the jejunum, such that feeding can bypass the stomach entirely.

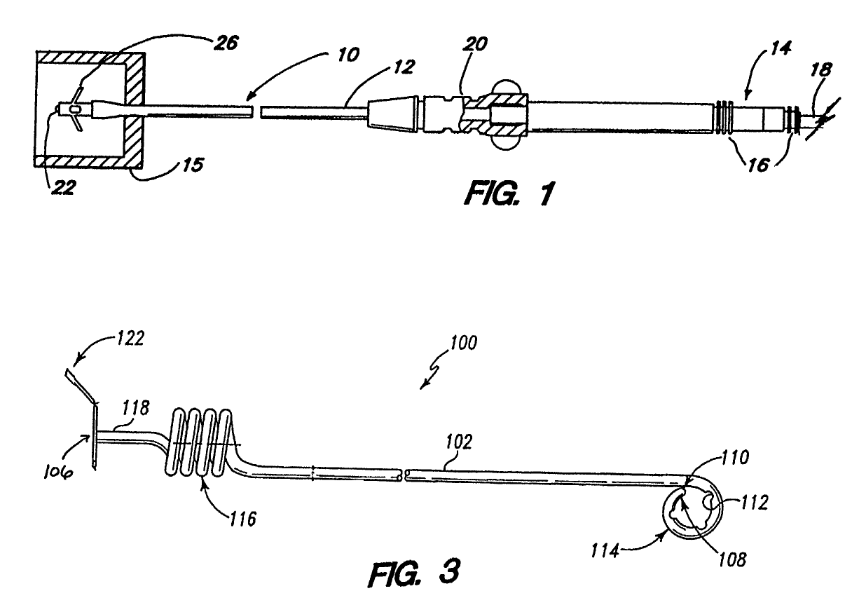

FIG. 3 illustrates an exemplary embodiment of an indwelling catheter, indicated generally at 100. The catheter 100 comprises an elongated tube 102 having at least one open central lumen 104 extending therethrough and a tissue-contacting surface. The tube 102 includes a polymer in intimate contact with an anti-inflammatory agent. The polymer of this tissue-contacting surface is fabricated of a flexible biostable polymeric material, such as polyurethane or silicone rubber, or combination thereof, or other polymers as described above. The proximal end of the tube 102 includes an opening 106 which communicates with the lumen 104. The distal end 108 of the tube 102 is preferably tapered and includes an axially directed end hole 110. Preferably, the tube 102 further includes a plurality of side ports 112 within the distal pigtail 114. The end hole 110 and the side ports 112 provide paths for fluid communication between the interior lumen 104 and the outside of the catheter 100.

In this embodiment, the catheter 100 includes a distal pigtail loop 114 so that the internal end of the catheter will be blunt and non-irritating. The proximal end of the catheter 100 includes a fitting 122, preferably in the form of a flange that sits substantially flush with the exterior surface of the patient's skin when the catheter 100 is in place. In this embodiment catheter 100 further includes one or more helically wound loops 116 near the proximal end of the catheter 100. A short, substantially straight section 118 of the catheter 100 lies between the helical loops 116 and the proximal end of the catheter. Both the distal pigtail 114 and the helical loops 116 are formed in the catheter 100 such that they will straighten out when a metal stiffener is inserted into the central lumen 104 of the catheter, and will then automatically reform when the metal stiffener is removed from the catheter 100 after placement of the catheter, such that the fitting 122 is held against the external surface of the patient and the at least one helical coil 116 is held against an interior surface of the cavity.

Indwelling catheters according to the present invention can be made by a variety of methods. In one embodiment, a method of making an indwelling catheter includes: providing an elongate body having a proximal end, a distal end, a tissue-contacting surface, and an interior lumen therethrough; wherein the tissue-contacting surface comprises a polymer in intimate contact with a steroidal anti-inflammatory agent; and coupling an external fitting to the proximal end of the elongate body. Preferably, the step of providing an elongate body comprises blending a steroidal anti-inflammatory agent with a polymer and forming a tissue-contacting surface. Alternatively, the step of providing an elongate body comprises coating a steroidal anti-inflammatory agent onto the tissue-contacting surface of the elongate body.

### EXAMPLES

The following examples are intended for illustration purposes only. All percentages are by weight unless otherwise specified.

### Example 1

### In vitro Biological Oxidation and Environmental Sress Cracking in Polyetherurethane

A. Materials and Methods

1. Cell Isolation

Human and rabbit blood was used as sources of the cells in these experiments. Blood was anticoagulated with 2 units/ml sodium heparin (Upjohn Co., Kalamazoo, Mich.). Mononuclear cells (lymphocytes, monocytes) were isolated within 15 minutes by a one-step density gradient centrifugation procedure using Isopaque-1077 (a density gradient solution) according to a modified Boyum's method (Boyum et al., Blood Separation and Plasma Fractionation, 217-239, Wiley-Liss, Inc. (1991)). The mononuclear cells were harvested and washed twice with cold Hanks balanced salt solution (HBSS) without Ca²⁺ and Mg²⁺ to minimize cell aggregation. The cells were then resuspended in standard media (RPMI-1640, 10% Fetal bovine serum, 0.2M L-glutamine, 10 Ul/ml Penicillin-G, and 0.1 mg/ml Streptomycin). The cell suspension was seeded into several plastic tissue culture flasks and incubated in the presence of 5% CO₂ at 37° C. for 1 hour (Ackerman et al., J. Immunol., 20, 1372-1374 (1978)). After this incubation, adherent (monocytes) were gently scrapped from the surface and resuspended in standard media. Nonadherent cells (lymphocytes) contained in the supernatant were recovered into sterile tubes, and the remaining nonadherent cells washed off with cold HBSS. The culture flasks were washed three times with cold HBSS, and the remaining adherent cells (monocytes) were gently scrapped from the surface and resuspended in standard media. Both cell types were resuspended to a density of 3×10⁶/ml.

2. Test Materials

Polymer discs, 6 mm in diameter, 0.12±0.008 mm thick, were cut out of polyetherurethane (PEU) sheets using biopsy punches (Prestwick Line, S.M.S. Inc., Columbia, Md.). One group of polymer discs were soaked in acetone (AS) for 1 hour to extract polymer antioxidants, and dried at room temperature for 4 hours. The other group was used with no pretreatment (non-AS). Polymer specimens were then fitted to the bottom of the wells of 96-microwell cell culture plates under sterile conditions.

3. In vitro Polymer Treatments

A 2-step in-vitro treatment was carried out at 37° C. to mimic the in-vivo environment and facilitate the biodegradation of the PEU sheets.

Macrophage Treatment.

The PEU film specimens (AS and non-AS) in the microwell plates were covered with either freshly isolated human or rabbit monocyte-derived macrophage (Mo/MØs), or human lymphocytes (3×10⁵ cells per well) and cultured in a standard media (RPMI-1640, 10% Fetal bovine serum, 0.2M L-Glutamine, 10 Ul/ml Penicillin-G and 0.1 mg/ml Streptomycin). A 49-day macrophage treatment was conducted under standard conditions (i.e., presence of 5% CO₂, and 95% humidity at 37° C.). Other experimental variations included adding dexamethasone sodium phosphate (DSP) at 0.024 μg/ml and 240 μg/ml concentrations to the culture media in the microwells. Two blank conditions were also studied. In one, culture media only was placed into the microwells. The other was prepared with no culture media and stored in the dark. All polymer specimens were incubated for the time of the first treatment. Samples in triplicate were removed after various time periods for hydroperoxide determination. After a 49-day incubation, specimens were prepared in triplicate for the second step of the sample treatment protocol.

FeCl₂ Treatment.

Following the 49-day treatment with macrophages, specimens were folded in half and fixed in this position by heat sealing the two opposite ends in such a fashion that an area of increased stress in the central region of the specimens. This design permitted a characterization of unstrained and moderately strained polymer states. Stressed specimens were incubated in 5 mM FeCl₂ at 37° C. for 10 days. Optical microscope (OM) evaluation of the samples was performed during the treatment. Triplicate samples for each condition were taken after 10-day treatment for scanning electron microscope (SEM) evaluation of the polymer surface.

4. Iodometry

Polymer specimens taken at various time periods during the macrophage treatment step were sonicated for 15 minutes in distilled water, rinsed three times, and dried at 25° C. for 4 hours. Hydroperoxide (ROOH) determination using an iodometric assay was performed as described by Fujimoto et al., J. Polym. Chem., 31, 1035-1043 (1993). This method is based on the reactivity of the hydroperoxide group, which oxidizes iodide to iodine. The resulting triiodide complex (I₃⁻) was measured spectrophotometrically at 360 nm λ with a Beckman DU-8 spectrophotometer (Beckman Instruments, Irvine, Calif.). This method measures the total (surface plus bulk) hydroperoxide concentration in the polymer.

5. Cell Morphology and Surface Analysis

The polymer films (0.12 mm thick) were sufficiently thin and transparent to enable visualization of cells on their surfaces during cell culturing using optical microscopy (OM) with an Olympus BX40 light microscope. For SEM cell morphology evaluation, specimens were taken after 21 days of macrophage cell culture. They were prepared for SEM evaluation by placing them into a cold fixative solution containing “PLASMA-LYTE” A (isotonic solution from Baxter Scientific, IL) and 1.5% glutaraldehyde. They were then stored at 4° C. for 48 hours. The samples were then removed from the glutaraldehydr fixative, rinsed in “PLASMA-LYTE” A three times for 15 minutes each. Following this, they were post-fixed with Palade's fixative (4% solution osmium tetroxide, Polysciences, Warrington, Pa.) for 2 hours. Following post-fixation, the samples were rinsed in “PLASMA-LYTE” A three times for 10 minutes each, and then slowly dehydrated using increasing concentrations of ethanol. They were finally critically point dried using CO₂. The polymer surfaces were also evaluated using SEM following the 10-day treatment with FeCl₂. All SEM specimens were mounted and sputter coated with gold-palladium for 2 minutes at 10 mA (≈100 Angstroms coating thickness), using a Humme IV Sputterer Coater (Anatech, Alexandria. Va.). Observation at different magnifications was done with a Stereoscan 360 (Cambridge Instruments) scanning electron microscope.

B. Results

1. Morphology of Mo/MØ Monolayers

The morphologic changes in the cell monolayer during the macrophage treatment step on the different surfaces were studied using OM and SEM analysis. Using OM analysis, early during culture, cells in the standard media started increasing their size, which continued to increase over time. The Mo/MØ monolayers in the standard media showed a variety of shapes, morphologies, and degrees of cytoplasmic spreading. The morphological changes that occurred between 0 and 33 days in these cells were extensive—increased size, cytoplasmic spreading, unusual shapes assumed with 60 μm diameter along the larger axis. A decrease in the number of cells was observed over time in the standard culture media. Mo/MØ monolayers cultured with DSP showed no increase in cell size; however, a few cells were observed to develop morphology similar to those cultured with standard media.

SEM analysis of 21-day cultured cells (standard media) showed a high degree of cell attachment and spreading of Mo/MØs on PEU. Cells were usually hemispherical with a central nucleus and extensive membrane ruffles indicating cellular activation. The dimensions of the cells varied between 25 μm and 60 μm depending on the degree and eccentricity of the spreading. In contrast, Mo/MØs cultured in the presence of 0.024 μg/ml DSP showed a smaller degree of cell spreading. The latter cells also showed numerous cytoplasmic processes (membrane prolongations). The nuclei of these cells tended to be fairly hemispherical, while the cells' surfaces, which often included protrusions, adopted a variety of shapes. These cells were highly variable in size, but were usually less than 35 μm along their larger axis. Other test conditions—human Mo/MØs cultured in the presence of 240 μg/ml DSP and human lymphocytes cultured in standard media in which a viable cell monolayer was observed under OM—showed no cells on the polymer surface when evaluated with SEM.

2. Polymer Hydroperoxide Evaluation

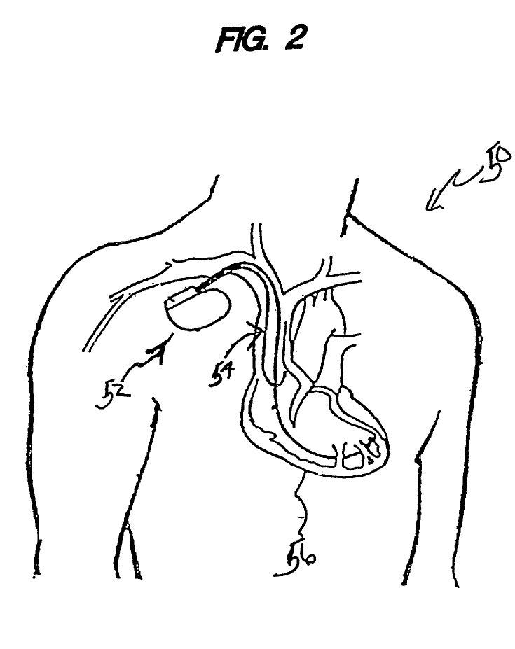

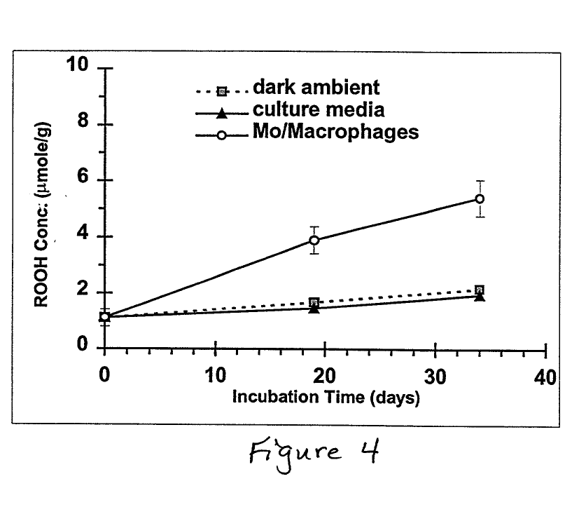

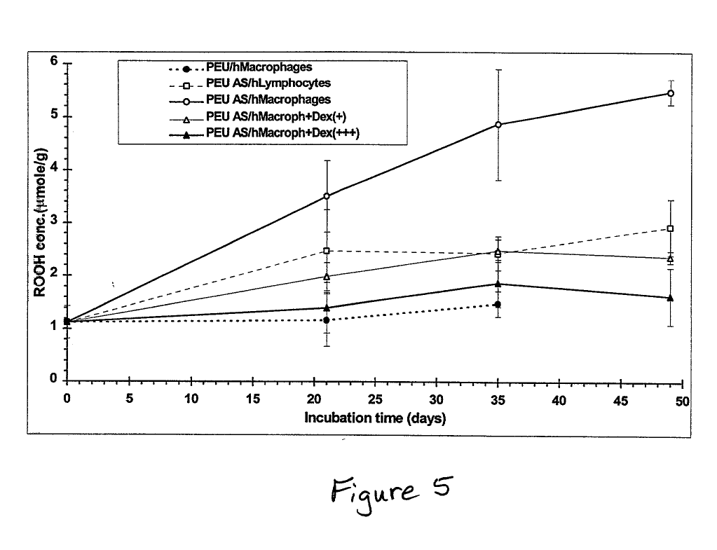

FIGS. 4 (rabbit) and 5 (human) show the hydroperoxide concentration in the polymer specimens treated under the different conditions described above. These conditions included: (1) standard culture media only (no cells); (2) polymer specimen stored in the dark under ambient conditions (without culture media); (3) human and rabbit Mo/MØs in standard culture media; (4) human lymphocytes in standard culture media; and (5) human Mo/MØs in standard culture media plus DSP at 0.024 and 240 μg/ml.

The data shows an increased hydroperoxide concentration as a function of culture time and the presence of Mo/MØs. This effect was marked in AS specimens (polymer specimens soaked in acetone before treatment) cultured with Mo/MØs from either source (rabbit or human) in standard media. By contrast, AS specimens cultured with lymphocytes or Mo/MØs in the presence of DSP showed significantly lower hydroperoxide concentrations. This was comparable to levels of hydroperoxide concentration in specimens incubated in culture media only and the ones stored in the dark under ambient conditions. Likewise, non-AS polymer specimens (not soaked in acetone before treatment) cultured with Mo/MØs showed the lowest amount of hydroperoxides.

3. Surface Analysis (SEM)

SEM examination revealed substantial pitting and cracking in the AS PEU samples exposed to Mo/MØs, with the stressed (folded) area as the more affected surface region. In this region, cracks up to 20 μm wide had developed. The cracks first initiated in pits to adopt a fibrillar structure and later propagated perpendicular to the applied strain direction caused by the folding. In contrast, specimens cultured with lymphocytes or DSP showed no significant damage. AS PEU samples exposed to Mo/MØs followed by FeCl₂ showed more extensive damage. In contrast, non-AS PEU samples exposed to Mo/MØs followed by FeCl₂ did not show appreciable surface damage. AS PEU samples exposed to Mo/MØs plus DSP followed by FeCl₂ showed only very occasional pits.

C. Conclusion

This study indicates that macrophages are involved in polyetherurethane oxidation, probably by inducing hydroperoxide formation in the polymer structure. Under the influence of stress or strain, polymers with sufficient hydroperoxides degraded in the presence of Fe²⁺ ions in a manner that closely resembles stress cracking observed in vivo. Likewise, a reduction in hydroperoxide formation and no later ESC development was demonstrated in macrophage-cultured PEU in the presence of DSP.

### Example 2

### In vitro Modulation of Macrophage Phenotype on Dexamethasone-Loaded Polymer and its Effect on Polymer Stability in a Human Macrophage/Fe/Stress System

A. Materials and Methods

1. Test Cell Line; Human Monocyte-Derived Macrophages (Mo/MØ)

The in vitro method used is described in Example 1. Human venous blood was used as the source of cells, which were isolated as described in Example 1.

2. Test Materials

Dexamethasone-Loaded “PELLETHANE” 80A (DEX/Pe80AS).

To prepare these materials, before extrusion, “PELLETHANE” 80A (Pe 80A, commercially available from Dow Chemical, Midland, Mich.) was extracted for 24 hours in a Soxhlett extractor using acetone. The purpose of this process was the removal of antioxidant from the polymer. After extraction, the material was dried under vacuum at 50° C. for 4 days. Dexamethasone USP Micronized BP/EP (Lot 78AFT, Upjohn Co.) was vacuum dried overnight at 40° C. In order to prepare materials with different dexamethasone (DEX) concentrations, the ratio of drug to polymer was varied to achieve 0.1% and 1% drug loading levels (w/w). Extrusion of 0.02-inch films was obtained at 0.1%DEX/Pe80A and 1%DEX/Pe80A) formulations.

“PELLETHANE” 80A Control (Pe80A).

Using the same “PELLETHANE” 80A polymer (acetone extracted), 0.02-inch films were extruded without DEX. Extrusion conditions with and without DEX were similar and were as recommended by the manufacturer.

Polymer discs, 6 mm in diameter, were cut out of the Pe80A test and control film sheets using biopsy punches. Polymer specimens (n=16 per condition) were then fitted to the bottom of the wells of 96-microwell cell culture plates under sterile conditions.

3. In vitro Polymer Treatments

A 2-step in-vitro treatment was carried out at 37° C., substantially as described in Example 1.

Macrophage Treatment.

The PEU film specimens (test and controls) in the microwell plates were covered with a freshly isolated human monocyte-derived macrophage (hMo/MØs) monolayer at a density of 3×10⁵ cells per well and cultured in a standard media (RPMI-1640, 10% Fetal bovine serum, 0.2M L-Glutamine, 10 Ul/ml Penicillin-G and 0.1 mg/ml Streptomycin). A 40-day macrophage treatment was conducted under standard conditions (i.e., 5% CO₂, 95% humidity, 37° C.). Freshly isolated hMo/MØs were added into the wells once a week. Immediately before the last cell refreshing, all wells were energically rinsed with culture media to detach and remove all cell components and remains, after which a fresh macrophage monolayer was applied. After this 40-day macrophage treatment, polymer samples were removed in triplicate for hydroperoxide determination and in quintuplicate for the-second step treatment.

FeCl₂ Treatment.

Following the 40-day macrophage treatment, specimens prepared and treated as described in Example 1.

4. Iodometry

Polymer specimens taken after the 40-day macrophage treatment step were sonicated for 15 minutes in distilled water, rinsed three times, and dried at 25° C. for 4 hours. Hydroperoxide (ROOH) determination using an iodometric assay was performed as described in Example 1.

5. Cell Morphology and Surface Analysis

OM observation of cultured cells was performed during the macrophage treatment step. Likewise, the stressed polymer surfaces were evaluated using SEM following the 10-day treatment with FeCl₂. The specimens for SEM evaluation were rinsed in distilled water and dried at room temperature. All SEM specimens were mounted and sputter coated with gold-palladium for 2 minutes at 10 mA (≈100 Angstoms coating thickness), using a Humme IV Sputterer Coater (Anatech, Alexandria, Va.). Observation at different magnifications was done with a “STEREOSCAN” 360 (Cambridge Instruments) scanning electron microscope.

6. Kinetics of DEX Elution from DEX/PEU Test Materials

DEX release profile from 0.1% DEX/Pe8OA and 1% DEX/Pe80A was determined in vitro at 37° C. in PBS. Each of the materials was run in triplicate. The procedure involved the immersion of four 15 mm diameter disks (0.3659±0.02 g) in 15 ml of phosphate buffer (Product No. P-4417, Sigma Chemical Co., St. Louis, Mo.). The average thicknesses of the disks were 0.47±0.06 mm. In a 32-day period at various timepoints, 800 μL of buffer was removed for analysis and replaced with fresh buffer to keep the elution volume constant. The aliquots were cold stored (4° C.) until analysis by HPLC.

7. HPCL Analysis

DEX was analyzed using reversed-phase chromatography and UV-visible detection. An octadecylsilane column (Product No. 07125, Tosohaas Bioseparations Specialists, Montgomeryville, Pa.) and mobile phase consisting of methanol and phosphate buffer (100 mM, pH 5.6) were chosen for this purpose. Furthermore, the flow rate (1.0 ml/minute) and use of detection wavelength, peak areas and autointegration remained constant for all experiments. From this data, a cumulative elution profile and a daily DEX elution was calculated.

8. Cytokine Analysis

In order to assess the in vitro expression of IL-1α and IL-8, human primary monocytes were incubated with various concentrations of DEX (2.5, 0.25, and 0.025 μg/ml) and methotrexate (50, 5, and 0.5 μg/ml). A higher rate of IL-1 and IL-8 inhibition was observed with these agents, with DEX having the highest levels of inhibition. The inhibition appeared to be dose- and incubation time-dependent. These results further support the anti-inflammatory ability and the effects of these agents on human macrophages.

B. Results

1. Morphology of Mo/MO Monolayers

The morphologic changes in the cell monolayer during the macrophage treatment step on the different surfaces were studied. A 100× OM observation through a Pe80A control film showed uniform cell distribution. The morphological changes that occurred between 1 and 40 days in these cells were extensive and showed to be different for each material condition. Human Mo/MOØ monolayers on the test surfaces (DEX/Pe80AS) and on control surfaces (Pe80A) at 3 days of culture evidenced little or no differences.

At later analysis, 20 days, noticeable differences were observed among the monolayers in the different surfaces. While a much higher proportion of macrophages with increased size and high degrees of cytoplasmic spreading were observed on Pe80A control material, Mo/MØs cultured on DEX/Pe80AS were observed to be roundly shaped with shorter diameters and with less density. Evaluation at 40 days of polymer treatment showed the same cell phenotype seen at 20 days, although a more marked effect, or cells with a maximum of 60 mm diameter along their larger axis on controls and up to 20 μm on test materials.

2. Polymer Hydroperoxide Evaluation

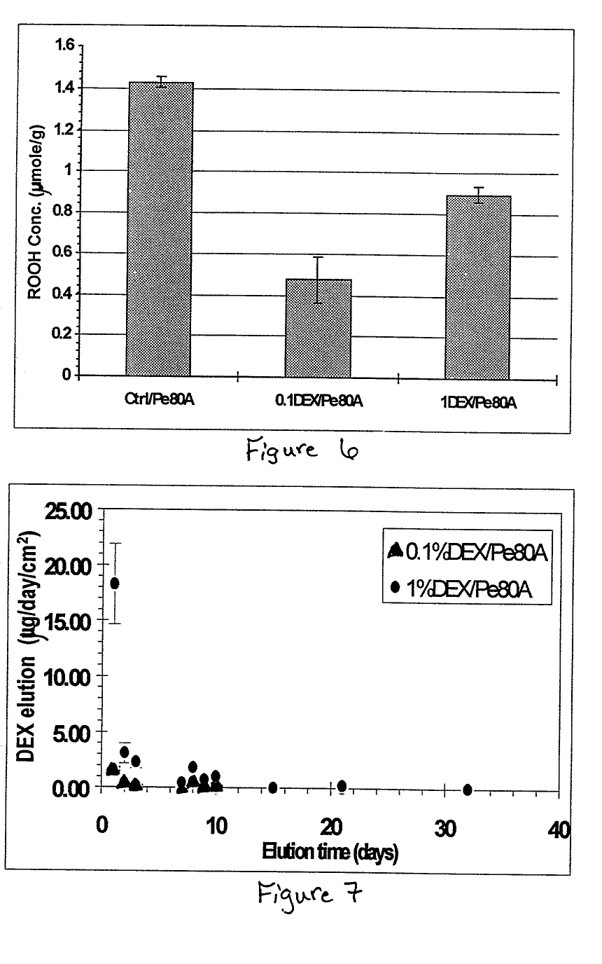

FIG. 6 shows the hydroperoxide concentration in the polymer specimens after the 40-day macrophage treatment step. The formation of ROOH in Pe80A followed a DEX-dependent effect. Significantly lower ROOH concentration in DEX/Pe80A specimens was observed. Thus, after 40 days of hMo/MOØ treatment, 0.5±0.1 and 0.9±0.04 μmole ROOH/g of polymer were contained in DEX/Pe80AS (0.1% and 1% w/w, respectively). By contrast, 1.4±0.02 μmole ROOH/g of polymer was contained in the control material.

3. Surface Analysis (OM and SEM)

During the FeCl₂ treatment, a daily OM observation of stressed polymer specimens was conducted at 40× total magnification in an Olympus SZH10 Research Stereo Microscope (Olympus Optical Co. LTD). Noticeable surface changes were evident starting at 4 days incubation in FeCl₂ at 37° C. At 6 days, well-developed pits and cracks were visible in the areas of major stress in all samples of Pe80A control material. Under the same conditions of treatment, both DEX/Pe80A specimens, 0.1% and 1%, showed a shiny surface with no apparent damage. In order to expand the damage in the test samples and to induce damage in test materials, the FeCl₂ treatment was extended up to 10 days, after which the samples were analyzed under SEM.

SEM examination revealed substantial pitting and cracking in the Pe80A control samples, with the stressed (folded) area as the more affected surface region. In this region, cracks up to 70 μm wide had developed. The cracks first initiated in pits to adopt a fibrillar structure and later propagated perpendicular to the applied strain direction caused by the folding. By contrast, none of the DEX/Pe80A specimens showed damage; rather, a smooth surface was observed in both DEX-containing specimens.

In an attempt to obtain semiquantitative data from this evaluation, an experimental X/Y rating system was adopted. In an X/Y system, which evaluates the depth of the cracks (X) and the extension of the surface affected by environmental stress cracking (ESC) damage (Y), the product is used to compare the different test conditions. The results can range from 0 to 25, with the lowest indicating the least damage. Table 2 shows the rating of the biostability evaluation of specimens following a 40-day treatment with human Mo/MØs and 10 days with FeCl₂. The final rating in this experimental scoring method is expressed as the average of the product of the X and Y values.

TABLE 2
In vitro Biostability Evaluation of DEX/Pe80A films

| | ESC Rating post 40-day MO/10-day | | | | | |
|---|---|---|---|---|---|---|
| | FeCl₂ treatment | | | | | |
| Material | | | | | | Final |
| Sample | 1 | 2 | 3 | 4 | 5 | Rating |
| Pe80A | 4/3 | 4/4 | 4/4 | 4/4 | 4/4 | 15.3 |
| 0.1% | 0/5 | 0/5 | 0/5 | 1/1 | 0/5 | 0.2 |
| DEX/Pe80A | | | | | | |
| 1% | 0/5 | 0/5 | 1/1 | 1/1 | 0/5 | 0.4 |
| DEX/Pe80A | | | | | | |

n=5. Final rating expressed as Mean. Observation at 70-100×.

Experimental rating=X/Y, X quantifies depth of cracks and Y quantifies extent of stressed surface coverage.

X=0 (no changes); 1 (change but no cracks, frosted areas); 2 (pits); 3 (cracks up to halfway through the film wall); 4 (confluent cracks); 5 (cracks 100% through the tubing wall, failure).

Y=0 (no changes); 1 (over <20% of surface); 2 (over >20 and <40% of surface); 3 (over >40 and ≦60% of surface); 4 (over >60 and <80% of surface); 5 (over >80% of surface).

4. Profile of DEX Elution from DEX/Pe80AS

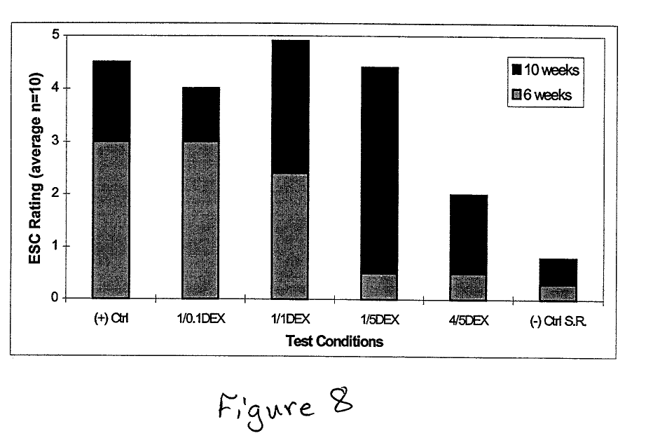

FIG. 7 shows the amount of DEX elution per material surface area (cm²) over a period of 32 days. Independent of the DEX loading in the material, an initial burst of DEX release was observed at day 1. As suspected, the amount of DEX release was directly dependent on the total DEX concentration in the polymer. At the first day, 1.6±0.2 and 19.5±0.4 μg of DEX was eluted per cm² of material (0.1% and 1% DEX/Pe80AS, respectively). This release declined sharply thereafter. From day 5 to day 32 there was a slowly decreasing level of elution. After this gradual decline, a release of 0.02±0.01 and 0.06±0.03 μg/day/cm² was registered at day 32.

C. Conclusion

This in vitro biological system has shown to be an effective tool for studying polymer degradation. The use of components that are present and available in the body during host responses (i.e., Mo/MØs, Fe, stress) make it a rather realistic method to replicate ESC degradation. These observations suggest that Fe⁺² ions accelerate hydroperoxide decomposition, resulting in a degraded polymer, and that the down modulation of macrophage's ability to generate reactive oxygen species through a controlled DEX release prevents the initial steps that lead to polymer degradation. The efficacy of this approach was demonstrated in this study by the reduction of hydroperoxide formation and no subsequent ESC damage in polyetherurethanes (Pe80A) loaded with DEX and treated in the Mo/MØ/Fe/stress system.

### Example 3

In vivo Biostability of Dexamethasone/Polymer Coatings in an Accelerated Test Model

A. Materials and Methods

1. Biostability Sample Configuration

Each biostability sample consisted of a piece of coated test tubing or control tubing strained to 400% elongation. Polysulfone mandrels were used to support the strained tubing. A 2-0 Ticron suture was used to sustain the strain of the tubing samples over the mandrels. The implant material strands consisted of five samples made specifically for test or control conditions. Each rabbit was implanted in the subcutaneous tissue of their backs with four, 5-sample strands. Each strand was identified by an attached glass bead whose color was coded to reflect the coating/control condition. The implant material strands measured approximately 0.3 cm in diameter and 7.0 cm in length. A total of 120 samples from 6 conditions were implanted in 6 rabbits, 20 per animal and 5 from each condition.

2. Test Coatings

Several formulations of DEX/Pe80A with varied DEX concentration were prepared. On the basis of DEX concentrations (w/w) the solutions were 0.1% DEX/Pe80A, 1% DEX/Pe80A, 5% DEX/Pe80A and Pe80A (w/o DEX). The solutions were prepared at 5% concentration of solids in THF and were used for dip coating of “PELLETHANE” 2363 80A tubing (Pe8OA, Dow Chemical Co., Midland, Mich.), c/c (cold/cold extrusion process), 0.070 inch ID×0.080 inch OD. For negative controls Pe 2363 80A tubing, h/h (hot/hot process), 0.070 inch ID×0.080 inch OD, was used.

Sections of the cold/cold Pe80A tubings were coated with the different DEX/Pe80A preparations by 1 or more dips as follows:

Pe80A c/c tubing coated (1 dip) with 0.1% DEX/Pe80A—resulting in about 2.4 μg/cm² DEX initially and about 0.6 μg/cm² DEX after 400% elongation (referred to herein as 1/0.1 DEX/Pe80A);

Pe80A c/c tubing coated (1 dip) with 1% DEX/Pe80A—resulting in about 22 μg/cm² DEX initially and about 5.4 μg/cm² DEX after 400% elongation (referred to herein as 1/1DEX/Pe80A);

Pe80A c/c tubing coated (1 dip) with 5% DEX/Pe80A—resulting in about 120 μg/cm² DEX initially and about 30 μg/cm² DEX after 400% elongation (referred to herein as 1/5DEX/Pe80A); and

Pe80A c/c tubing coated (4 dips) with 5% DEX/Pe80A—resulting in about 373 μg/cm² DEX initially and about 93 μg/cm² DEX after 400% elongation (referred to herein as 4/5DEX/Pe80A).

All the samples were sterilized with one cycle of ethylene oxide as is well known in the art.

3. Control Coatings

For positive controls, Pe80A-coated (1 dip) Pe 2363 80A tubing, c/c, 0.070 inch ID×0.080 inch OD was used. For negative controls, non-coated Pe 2363 80A tubing, h/h, 0.070 inch×0.080 inch OD, was used. Biostability samples in this condition were stress relieved (S.R.) at 150° C. for 15 minutes. All samples were prepared at 400% strain. The controls were sterilized with ethylene oxide.

4. Test Animals

Six (6) healthy adult male or female New Zealand white rabbits were used. All test and control biostability samples were implanted under general anesthesia. A total of 20 biostability samples were implanted in each animal. Due to the potential cross effect of dexamethasone, two animals were implanted with controls and four animals with DEX-containing samples. The individual samples were assembled into strands, with five samples per strand. Each strand had a colored glass bead to identify each experimental condition. They were implanted in the subcutaneous tissue in the backs of rabbits. Two strands were implanted on the left side of the spine parallel to the dorsal midline. Two strands were implanted on the right side of the spine parallel to the dorsal midline. Euthanasia and explantation of the samples were conducted at two timepoints, 6 and 10 weeks (10 samples per condition and per timepoint).

5. Accelerated Biostability Test Model

An accelerated in vivo biostabiltiy model was used. Sections of test and control tubings were prepared at 400% elongation. The negative control (Pe80A h/h) was stress relieved at 150° C. for 15 minutes. After one cycle of ethylene oxide sterilization, the sample strands were implanted.

6. Sample Analysis

Upon termination of the rabbits, the samples were explanted. No abnormal tissue response at the implant sites was noted macroscopically. The samples were debrided of tissue and rinsed in distilled water. After being dried, the samples were examined by optical microscopy at up to 70× without further sample preparation. For analysis, the samples were rated for environmental stress cracking in a manner similar to that described in Example 2 (Table 2). Each individual rating was slightly different, however, the ranges of values for X and Y were similar (X=0 (no changes) to 5 (cracks 100% through the tubing wall, failure) and Y=0 (no changes) to 5 (over >80% of surface).

B. Results

At the end of the 6 and 10 post-implanatation week, 3 animals per timepoint were euthanized and the samples explanted. The explanted samples were debrided of tissue and dried for optical microscopy (OM) evaluation. Representative samples were also evaluated by scanning electron microscopy (SEM) (samples were dried, mounted, and sputter coated with gold palladium as described in Example 1). Under OM, the samples were inspected for defects and flaws.

The overall results showed the following:

1. Positive control (worst case), Pe8OA (NoDEX). At 6 weeks, 4 samples showed ESC failure (5/1 score), with shallow cracks and near failure observed in 3 samples. At 10 weeks, ESC failure occurred on all but 2 samples.

2. 1/0.1DEX/Pe80A Test coating (0.6 μg DEX/cm²). At 6 weeks, 6 samples showed ESC failure, and four showed no changes. At 10 weeks, 6 samples failed and 3 showed no ESC changes.

3. 1/1 DEX/Pe80A Test coating (5.4±0.7 μg DEX/cm²). At 6 weeks, 4 samples showed failure, 1 near failure, and 5 samples with no ESC changes. At 10 weeks, all samples except one showed ESC failure.

4. 1/5DEX/Pe80A Test coating (30±0.6 μg DEX/cm²). At 6 weeks, no failed samples were encounted. At 10 weeks, 6 samples showed ESC failure.

5. 4/5DEX/Pe80A Test coating (93.1 μg DEX/cm²). At 6 weeks, none of 10 samples showed ESC failure. At 10 weeks, 4 samples showed ESC failure. The remaining 6 samples had no ESC present.

6. Negative Control (best case), Pe80A h/h S.R. (stress relieved). At 6 weeks, no ESC was found on 8 samples, while 2 samples showed minimal changes and shallow cracks. At 10 weeks, 4 out of 10 samples had shallow ESC present. The remaining 6 samples has no ESC present.

In some of the 1-dip coated specimens, oval areas of defective coating were observed. This defect seemed to correlate with ESC damage (cracks and shallow cracks) in the area.

The protective mechanism appears to be effective as long as an adequate amount of DEX is present in the coating. This is evidenced by the clear DEX dose-dependency of the results. FIG. 8, which depicts a summary of the highest score in terms of ESC rating per specimen and per timepoint, graphically shows that while coating with 30 μg/cm² DEX (1/5DEX/Pe80A) was effective in preventing surface damage up to 6 weeks, an extensive damage, similar to the positive control condition was observed at 10 weeks. In contrast, coatings containing 93.1 μg/cm² DEX (4/5DEX/Pe80A) performed better than the positive control at both timepoints.

C. Conclusion

This study shows that dexamethasone has a protective effect on biodegradation of polymers and prevents the development of environmental stress cracking in oxidation-susceptible polyurethane.

### Example 4

### Anti-Inflammatory Devices: In vivo Studies

A. Materials and Methods

1. Test Animals

The animals used for implantation were 3-month-old, 250-300 g body weight, female Sprague Dawley rats purchased from Charles River Laboratories, Wilmington, Mass.

2. Cage Test System

The metal wire mesh from which the cages were made was type 304 stainless steel with a mesh size of 24, a wire diameter of 0.254 mm, and interstices measuring 0.8 mm×0.8 mm (Cleveland Wire Cloth and Manufacturing Co., Cleveland, Ohio). The dimensions of the cages were approximately 3.5 cm long and 1.0 cm in diameter. Each cage contained a piece of the control or test material of interest. Empty cages were used as test controls. These cages were packaged and sterilized with ethylene oxide as is well known in the art.

3. Test Materials

Dexamethasone-Loaded Polyurethane. A segmented aliphatic polyurethane as described in U.S. Pat. No. 4,873,308 (Coury et al.) with no additives was loaded with micronized, free base dexamethasone USP (DEX, Upjohn Co.) using a cosolvation process. The appropriate amount of DEX was dissolved in tetrahydrofuran (with no butylated hydroxytoluene), Aldrich Chemical Co., Milwaukee, Wis.), followed by the polymer. The solutions contained 14% solids and 1% and 20% DEX. The solution was cast in 9.5 cm×9.5 cm “TEFLON” trays. The 20% DEX-containing film was dried in a freezer at −17° C. for 4 days and then in a vacuum oven at 50° C. and −30 inches Hg for 2 days. The 1% DEX-containing film and control film (no DEX) were dried under ambient conditions for 1 day, at 50° C. for 4 days, and then at 50° C. and −30 inches Hg for 3 days. The dried 20% film had a thickness of 0.7 mm, and the 1% film and control film had thicknesses in the range of 0.44 mm to 0.62 mm. Specimens weighing 24.97±0.04 mg (control), 24.98±0.05 mg (1D), and 25.01±0.06 mg (20D) were prepared, placed in cages, and sterilized with ethylene oxide.

4. Implantation Procedure

One cage was implanted subcutaneously on each of the right and left sides of anesthesized test animals. For implantation purposes, the 33 rats were divided into 2 groups. In the first group, 15 animals were implanted. In the second group 18 animals were implanted. A 1.0-cm to 1.5-cm incision was made int he skin about 2 cm above the tail and along the midline. A pocket was made in the subcutaneous space just below the right or left shoulder blade using blunt dissection. A cage specimen was then inserted through the incision and positioned at the level of the panniculuc carnosus, with the seam placed against the underlying muscle. Another cage specimen was implanted on the other side of the rat in the same fashion. The skin incision was closed with clips (Fisher Scientific, Pittsburgh, PA). The closed wound was then sprayed gently with Betadine solution.

5. Exudate Analysis

Exudate was aspirated with syringes from the cages at days 4, 7, 14, and 21 post-implantation. To avoid interference with the body's inflammatory response, no more than 0.3 ml of exudate was collected from each cage at each time period. Total and differential cell counts were performed by personnel with no information about the exudate's identification using standard techniques. After the 21-day exudate sampling, the rats were euthanized by carbon dioxide asphyxiation.

6. Total Cell Count

To screen for the presence of infection, an aliquot from each exudate sample was cultured on 5% sheep's blood agar plates. Immediately after the exudate was withdrawn at days 4, 7, 14, and 21 post-implantation, the total cell count for each exudate was determined by hemocytometer counting.

7. Differential Cell Count

An aliquot of the exudate that contained approximately 15000 white blood cells (leukocytes) was transferred to a test tube with 300 ml RPMI-1640. Aliquots (200 μL) of the cell suspension were spun down onto a clean glass microslide using a cytocentrifuge (Shandon Inc., Pittsburgh, Pa.). These microslides were stained with “DIFF-QUICK” stain (Baxter Scientific, McGraw, Ill.) according to the manufacturer's recommendations and used for a quantitative differential cell count. Polymophonuclear (PMNs), monocyte-derived macrophages (Mo/MØs), and lymphoctyes were the cell types counted for this analysis.

8. Cage Analysis

Following the 21-day exudate wighdrawal, the implanted cages were removed from the euthanized animals and immediately evaluated macroscopically. The top edge of the cage was cut with a pair of scissors along the inner surface seam. Intact and opened cages were examined and described. After analysis, the cages were immersed into 1-% formalin jars.

To assess the amount of fibrous tissue in the explanted cages, the cages were dried at 60° C. for 72 hours and their dry weight was recorded. Following tissue digestion by cage immersion in 6N KOH for 2 hours at 80° C., the weight of each stainless steel cage was again recorded. Dry tissue weight (dry tissue/(total cage weight—cage's stainless steel weight) per cage was calculated.

9. Material Surface Analysis

Polymer specimens were retrieved with tweezers where possible, rinsed in “PLASMA-LYTE” A (Baxter Scientific, McGraw Park, Ill.), and placed onto a microslide. The specimens were then cut into two pieces with a razor blade. One piece was placed into a cold fixative containing “PLASMA-LYTE” A and 1.5% glutaraldehyde and stored at 4° C. The other piece was placed into an alcohol fixative and subsequently stained with “DIFF-QUICK” stain.

The polymer films (0.6 mm thick) were sufficiently thin and transparent to enable visualization of stained adherent leukocytes using optical microscopy (OM) with an Olympus BX40 light microscope. The stained polymer specimens were initially characterized on both sides, which were very similar, with numerous leukocytes adhering to each surface. Every cell attached to the substrate surface was counted differentially at 45×. Each foreign body giant cell (FBGC) was counted as one cell; although the number of nuclei contained within each FBGC was also recorded.

For SEM evaluation, specimens were removed from the glutaraldehydr fixative, rinsed in “PLASMA-LYTE” A three times for 15 minutes each and prepared as described in Example 1.

10. Statistical Analysis

The data is presented as the mean±SD. For total cell counts the unpaired Student's t test at 95% level of confidence (p<0.05) was used to compare group means. Test materials 1D (1% DEX) and 20D (20% DEX) were compared to the PU control film made using THF (A) and an empty cage (EC).

B. Results

1. Exudate Analysis

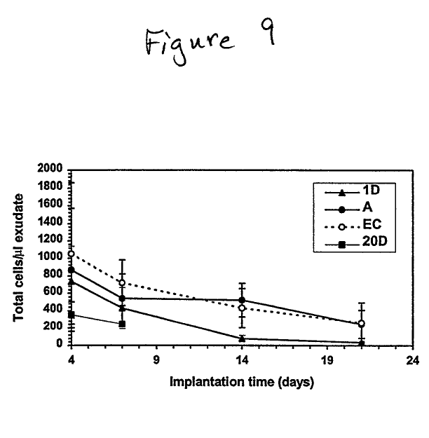

The leukocyte densities in the exudate samples drawn from the differential materials at 4, 7, 14, and 21 days post-implantation are displayed graphically in FIG. 9. A gradual decrease in cell density after 4 days was evident in all test conditions. DEX-containing materials (1D and 20D) clearly elicited lower cell numbers during the entire implantation time. This effect was statistically significant at 14 and 21 days for 1D and at 7 days for 20D.

At 4 days, 1D elicited 90% and 20D elicited only 40% of the cell number that was elicited in the control material (A). At 21 days, 1D exudates contained only 13.9% of the number of cells observed in exudates from the control polyurethane. Unfortunately, analysis for 20D material stopped at 7 days because of infection. Comparison of the control materials showed that the choice of solvent (THF and NMP) used in preparation of the PU film produced an effect on the results.

All cell types in the exudate, which included PMNs, macrophages, and lymphocytes, decreased over time post-implantation. At day 4, the leukocytes were dominated by polymorphonuclears (PMNs) and macrophages (Mos). At later time points, however, there was a rapid decline in the percentage of PMNs, reflecting the establishment of a chronic inflammatory response. While the concentration of the three leukocyte cell types in the exudate decreased with time, the considerable decrease in PMNs provided for the percentage increases observed for the two mononuclear cell types. Only macrophages and FBGCs were present on material surfaces at 21 days, although macrophages, lymphocytes, and PMNs were characteristically observed in exudates.

The total exudate cell count for test materials containing 1% DEX (1D) and 20% DEX (20D) elicited lower cell counts control materials A and EC, evidencing that they have significantly less potential to elicit inflammation. This effect was sustained throughout the study for 1D. The low PMN numbers observed at 14 days (approximately 5 cells/μL exudate) and 21 days (approximately 0.5 cell/μL exudate) in the 1D exudates, suggests that there was little or no influx of newly recruited PMNs to the inflammatory site. In other words, a mildly chronic inflammatory status prevailed after the acute phase had concluded. By contrast, PMNs were still present at 14 and 21 days in exudates from controls A and EC, which indicates that dexamethasone may accelerate the process toward a full wound-healing response.

2. Material Surface Analysis

Dramatic macroscopic differences were observed between DEX/PU-containing cages and other cages. Fibrous capsule formation was significantly lower in 1D cages (40.6±10.6 mg dry tissue per cage) than in control A cages (218.1±72 mg dry tissue per cage) or empty cages (207.9±70.7 mg dry tissue per cage). This shows the effectiveness of dexamethasone in reducing collagen production at the tissues surrounding an implant.

Surface analysis of materials after 21 days of implantation showed adherence of cells of the macrophage lineage. Under light microscopy at 45×, the majority of adherent cells were readily identified as FBGCs, although some of the observed cells showed classic macrophage morphology.

Different densities of adherent leukocytes were present on the surfaces. Most of the surfaces evidenced a more or less random cell distribution; however, there were areas of high cell population density, areas of scattered cells and occasional cell aggregates, and areas of very few cells. Adherent leukocytes evidenced varied morphologies and degrees of cytoplasmic spreading. Some of the cells had assumed unusual shapes, and some exhibited a deterioration of the cellular membrane, resulting in considerable effacement of the cell architecture. By day 21, some cell debris was present on all surfaces, except on 1D material. Since no surface analysis was done at earlier timepoints (e.g., 4, 7, 14 days), the progression in the process of cell distribution/adhesion was not explored.

Stained surfaces of 1D material evidenced a greater macrophage to FBGC ratio on their surfaces. On these surfaces, several macrophages and only scattered FBGCs were observed. In contrast, a considerable number of FBGCs and only occasional macrophages were present on the control material A (polyurethane film made with THF and no dexamethasone).

C. Conclusion

This study shows that dexamethasone-loaded polyurethane is effective at reducing inflammation in response to biomaterial implantation.

### Example 5

### In vivo Evaluation of Dexamethasone-Coated Transvenous Pacing Leads

A. Materials, Methods, and Results of DEX-Treated Pacing Leads

1. Preparation of Lead Prototypes

A set of experiments was designed to test the feasibility of coating pacing leads with DEX-loaded PU formulations. A segmented aliphatic polyurethane as described in U.S. Pat. No. 4,873,308 (Coury et al.) was loaded with DEX through cosolvation in THF as described in Example 4. The ratio of drug to polymer was varied to achieve either 1% or 5% drug-loading levels in solution. The appropriate amount of drug was first dissolved in THF. The polymer was then added and allowed to dissolve in the solution. At completion, the solutions were 11% solids (w/w). Under a filtered laminar flow hood, transvenous pacing leads Model Nos. 4023 and 4523 (Medtronic Inc., Minneapolis, Minn.) were weighed and then dipped into the DEX/PU/THF solutions. In order to vary DEX-loading in the devices, the solutions contained 0%, 1%, and 5% of DEX (w/w), at 11% wt/wt total solids. A control included only a coating of PU (11% solids).

A dipping device was configured to control the speed of immersion of the leads into the solution. Prior to the coating, the electrode tips and tines were protected with a piece of polypropylene tubing and parafilm. To facilitate the immersion of the leads into the DEX/polymer solution, a silicone coated 6.5 g. round split shot sinker (Water Gremlin Co., White Bear Lake, Minn.) was attached distally to each lead. Leads were then lowered into the coating solution to a depth of 15 cm (lead body) at a speed of 1.9 cm per second and then immediately lifted from the solution. Between dips the coated leads were left for at least about 4 hours in a forced-air oven (80° C.) and then vacuum dried (−30 inches Hg) for at least about 24 hours.

The total weight of the coatings on the devices increased with each additional dip, showing a good weight-to-dip linearity. In order to obtain a varied range of total DEX loading on these devices, the coating was considered completed following 2 dips for control PU-coated leads, 3 dips for 1%DEX/PU coated leads, and 4 dips for 5%DEX/PU coated leads. After dipping, the devices were released from their electrode/tines protection and trimmed under microscope. Final DEX content was determined by weighing each lead.

Dip coating leads in the DEX/PU solutions resulted in the deposition of a homogeneous polymer layer on the body surface. On the basis of DEX content per lead, three conditions were prepared. The final DEX loading is shown in Table 3. The coated devices were packaged and sterilized in ethylene oxide before their use for elution studies or for canine implantation.

TABLE 3
DEX Loading on Coated Leads (15 cm)

| | 1% DEX/PU (“Low”) | | 5% DEX/PU (“High”) | |
|---|---|---|---|---|
| Drug loading | Atrial | Ventricular | Atrial | Ventricular |
| Total DEX | 0.5 ± 0.1 | 0.4 ± 0.1 | 3.4 ± 0.2 | 2.8 ± 0.1 |
| (mg) | | | | |
| DEX/cm² S.A. | 0.09 ± 0.02 | 0.08 ± 0.02* | 0.6 ± 0.04 | 0.5 ± 0.01* |
| (mg) | | | | |

Note:
Data expressed in mg, mean ± SD, n = 3, *n = 7

2. Kinetics of in vitro DEX Elution from DEX-Coated Pacing Leads

The in vitro profile of DEX release from the two DEX-containing lead conditions, 1%DEX/PU and 5%DEX/PU (“Low” and “High” DEX loading respectively) was determined through elution experiments carried out at 37° C. in PBS.

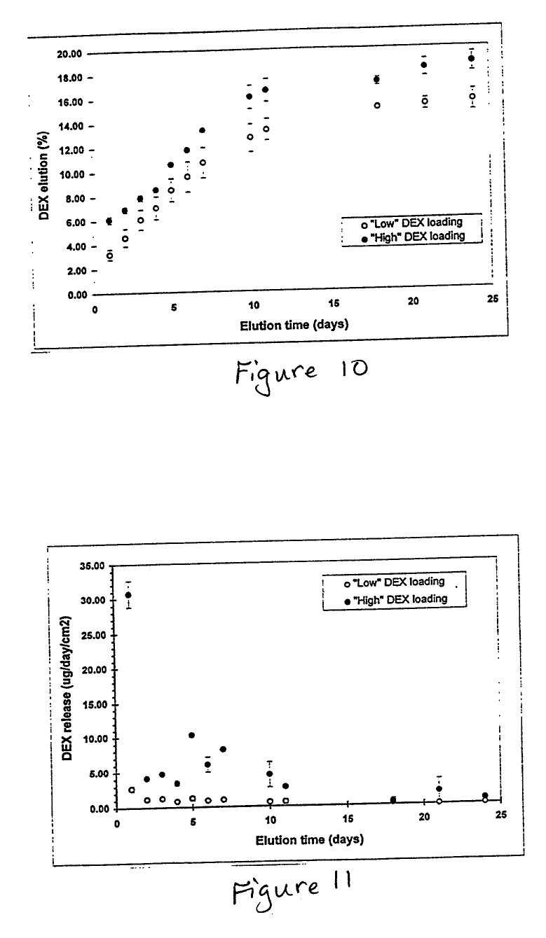

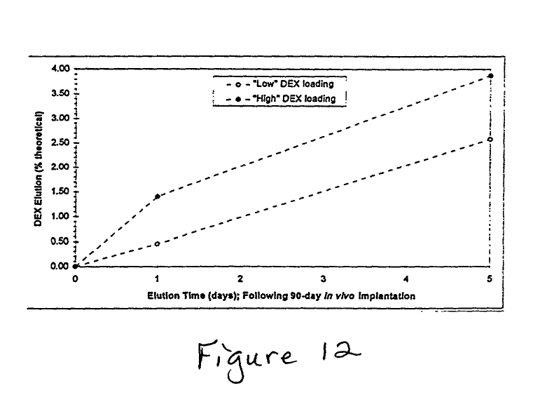

The coated portion (15 cm) of two leads from each condition were used for these analyses. Following the separation of the electrode/tines portion from the lead body (to remove the dexamethasone in the electrode), the coated lead bodies were immersed in PBS at 37° C. and the eluates were analyzed using HPLC as described in Example 1 at different time points within a 24-day period. FIG. 10 shows the cumulative DEX elution over time, expressed as the percentage of the total DEX loading per lead. Both lead conditions evidenced similar profiles of DEX elution. Following an accelerated elution lasting up to 10 days, it was observed that the elution slowed down. At 24 days, 15.5% and 18.7% of the total theoretical DEX loading was eluted from the “Low” (1%DEX) and “High” (5%DEX) DEX coated leads, respectively. FIG. 11, shows the amount of DEX elution per material surface area (cm²) per day over a period of 24 days. In an initial burst of drug release, 2.7±0.4 μg and 30.8±1.9 μg of DEX was released per cm² of leads coated with the “Low” and the “High” DEX conditions, respectively. The release of DEX declined sharply thereafter. From day 4 to day 24 there was a gradual decline in DEX release. At the 24th day of this experiment, 0.07±0.09 μg and 0.7±0.1 μg of DEX was released per cm² for the “Low” and the “High” DEX conditions, respectively. Although in vitro elution rates may be significantly different from the elution rates in vivo, these studies were useful in monitoring and validating the DEX loadings and the elution profiles of DEX coated-devices.

This release profile and elution rate (data not shown) were similar to that obtained with materials used in the in vivo cage study in Example 4. By varying the DEX concentration in the coating solutions, the percentage of solids in the coating solutions, or the number of dips, a useful biological range of DEX loading was achieved. This demonstrates the feasibility of obtaining coatings that exhibit desired loadings and release profiles of DEX for specific device applications. Of course dip-coating may not be the only method for applying this technology to leads and other devices. It is possible that extrusion and/or co-extrusion of DEX/PU materials could be used.

B. Materials, Methods, and Results of In vivo Evaluation of DEX-Coated Pacing Leads

1. Test Animals

The animals used for implantation were canines of random sex and with ≧25 kg body weight.

2. Lead Implantation

Three conditions of coated pacing leads were implanted into canines. As shown in Table 4, two DEX/PU coated lead conditions (“Low” and “High” DEX loading) and one PU-coated lead condition (control) were implanted in 6 canines. For this study, 3 (2 ventricular and 1 atrial) leads from test or control treatment conditions were implanted per dog. A 3-lead-per-dog model was adopted to increase the amount of hardware within the intracardiac chambers. The number of animals and specimens per material/condition are displayed in Table 4.

TABLE 4
Experimental Distribution of Animals and Coated Lead/Conditions

| DEX | Coating | | |
|---|---|---|---|
| Loading | Condition | No. Canines | No. Leads |
| “LOW” | 1% DEX/PU | 2 | 6 |
| “HIGH” | 5% DEX/PU | 2 | 6 |
| NO DEX | PU | 2 | 6 |
| Totals | 3 | 6 | 18 |

The ventricular leads were implanted through a 3rd intercostal right thoracotomy via costo-cervico-vertebral trunk (CCTV). The atrial leads were implanted through a right jugular venotomy. With the aid of fluoroscopy, one ventricular lead was placed in the RV apex and the other ventricular lead was placed in the RV posterior wall at least 1 cm from the apical lead. Thresholds of less than 1.0 V at 0.5 ms verified adequate ventricular and atrial lead placement using the Model 5311 Patient System Analyzer (Medtronic Inc., Minneapolis, Minn.). After securing leads in the vessels, the lead connector ends were tunneled to the right chest wall and capped with IS-1 pin caps. Lead placements were further documented with lateral and dorso-ventral X-ray analyses. In general, the surgical and post-surgical activities evolved without complications. Due the nature of this study, no steroid medications were administered to any canine.

3. Evaluation of Systemic Parameters

The regulatory influence of circulating steroids during the in vivo stage of implantation was evaluated. Severe depression of circulating levels of monocytes has been reported using (0.6 mg/g body weight, s.c.) hydrocortisone. Steroids can also depress the circulating level of T-lymphocytes in mice, rats, and humans. Likewise, steroids, particularly at immunosupressive doses, reduce the resistance to bacterial infection. Infections, when present, can be detected by changes in the differential distribution of blood cells or by positive bacterial culturing.

In order to evaluate systemic changes that might be attributable to DEX release (i.e., excessive corticosteroid, infection, etc.) from the treated devices, blood and hemogram analyses were performed at weeks 1, 2, 4, 8, and 12 post implant. At least one of these analyses was performed in the pre-operatory. Results showed that lymphocyte numbers were either within normal range or slightly elevated. In summary, a consistent or progressive finding of lymphopenia and/or eosinopenia was not noted in any dog on the study.

4. Intracardiac Macroscopic Pathology Evaluation

After 13 weeks (3 months) of lead implantation, the animals were heparinized, X-rayed, and euthanized following a standard procedure. Necropsy was performed by a pathologist who was kept blind to the different conditions in the study. Special emphasis was focused on the intracardiac compartment to evaluate lead-tissue relationships, encapsulation of the devices, their extension, thickness etc.

Following euthanasia, the heart was dissected, opened, and carefully removed. Right heart cavities were opened through a longitudinal incision to expose the implanted leads. Low and high magnification photographs were taken prior to and after opening the heart and after heart removal. After complete analysis and description of the findings, the hearts were placed in 10% buffered formalin.

With minimal differences among the dogs, the proximal ends of the three leads were surrounded by fibrous tissue in the subcutis over the right thorax. In general, two leads (ventricular) entered the thorax directly through the right thoracic wall and then to the venous system through the right CCTV at a venotomy site. One lead (atrial) traveled anteriorly through the subcutis over the right scapula to the right ventral neck, where it entered the jugular vein through a venotomy site. Subcutaneous sheaths were thin and tightly apposed to the leads.

Dog Receiving Leads Without DEX.

Two foci of soft yellow multifocal endocardial thickening (12×4 and 0.5 mm diameter respectively) were found dorsal to the intervenous tubercle. Atrial lead. A short translucent tissue sheath (<1 cm) surrounded the lead immediately distal to a secure jugular venotomy site. Its implantation site was verified to be within the right atrium appendage (RAA) in which a uniform, smooth, and shiny tissue sheath (8 mm long) was observed. The rest of the body lead was free of any adherent tissue from the level of the CCTV to its implant site in the right atrium appendage (RAA). Ventricular leads. Immediately distal to the CCTV venotomy site, a tissue sheath (4 mm long) covering the two leads was observed. This tissue sheath was complicated distally by the presence of an eccentric (5 mm long) antemortem thrombus. Within the anterior vena cava (AVC), right atrium (RA), and right ventricle (RV), both leads were predominantly free of adherent tissue. However, within the AVC, both leads were in a common tissue sheath, which was in turn attached to the luminal wall of the roof of the AVC. This short tissue had smooth translucent and uniform characteristics, and covered 7 mm and 9 mm of the two leads, respectively. The lead that was implanted more on the RV wall was adhered to the free wall of the RV by a lateral attachment. This tissue sheath with a trabecular muscle and smooth, shiny translucent tissue characteristics covered the lead for 12 mm.

Dog Receiving Leads Without DEX.

Atrial. Implant site was secure and located on the free wall of the RAA. Ventricular. Immediately distal to the CCTV, both leads are in a common tissue sheath which bifurcates slightly at its distal end. This tissue sheath was extended distally from the CCTV venotomy site and covered 1.2 cm of the apical lead and 1.1 cm of the wall lead. The apical lead was adhered to the tricuspid valve apparatus over a distance of approximately 1.5 cm. The most distal end of the apical lead was not visible. Implant site for the apical lead was secure. The caudal portion of the parietal leaflet of the tricuspid valve has its margins thickened by soft yellow tissue.

Dog Receiving Leads Coated With 1%DEX/PU.

Atrial lead. At the junction of the AVC and the crest of the RAA, there was a prominent soft endocardial thickening. The lead was securely implanted on the free wall of the RAA. Within the dorsal AVC, there was a multifocal yellow firm nodular endocardial thickening. Each nodule (2-3 mm diameter) were distributed over an area approximately 1.5 cm×1.0 cm. Ventricular leads. CCTV venotomy site was secure. Both leads, immediately distal to the CCTV venotomy site, were in a common tissue sheath with a mild thrombus on it. Apical and wall leads passed from the CCTV and was free of any tissue or material adhesion until it reaches the tricuspid valve, and present an adhesion to the parietal leaflet of 5 mm long. At this adhesion site, the margin of the valve was thickened by a firm nodular smooth shiny tissue. Distal to the tricuspid valve, the leads were free of adherent tissue or material until reached the RVA and the interventricular septum, in which showed a secure fixation. RVW lead this the lead is free of any adherence of tissue or other material until it reaches its implant site in the RV apex. The other lead (RV wall) has immediately to the CCTV venotomy site, two pale nodules of adherent tissue or material, each <1 mm diameter and a very transparent tissue sheath over a length of 3 mm. Approximately 7 cm distal to the CCTV venotomy site, two segments of tissue sheaths (5 mm and 2 mm long respectively) were observed, neither sheath was adhered to adjacent cardiac tissues. This lead passed through the tricuspid valve at its caudal commisure, no adhesions were observed. Focal soft yellow 6×3 mm endocardial thickening was noted on the RV free wall.

Dog Receiving Leads Coated With 1%DEX/PU.

Atrial. Immediately distal to the jugular venotomy site, the lead was within a 1.5 cm long smooth and shiny tissue sheath of variable thickness. This tissue sheath was complicated distally by an organized antemortem thrombus that was 0.5 cm long. This lead was securely implanted in the RAA. Multiple soft, smooth, and shiny endocardial thickenings on the roof of the AVC, on the anterior surface of the intervenous tubercle and at the junction of the RAA with the RA and the AVC were observed. The RA endocardium adjacent to the origin of the RAA was thickened by a pale opaque tissue. Ventricular Immediately distal to the CCVT venotomy site, both leads were in a common fibrous sheath 1.0 cm long that was complicated distally with a 1.0 cm long organized thrombus. One lead was noted to pass into the os of the coronary sinus and into the middle cardiac vein. The middle cardiac vein was opened, and the lead was found to be ensheathed over the distal 6 cm of the lead. The other lead passed from the right atrium into the RV, where it was securely implanted near the RVA. This lead passed through the tricuspid valve near the caudal commissure; it remained free of any adhesions to the valve apparatus. The distal end of this lead was buried within the trabecular muscle. The leaflets of the tricuspid valve had a soft, smooth, and shiny thickening in areas apposed to the RV lead.

Dog Receiving Leads Coated With 5%DEX/PU.

Within the anterior mediastinum, located at the thoracic inlet and adhered to the right first rib, an encapsulated gauze sponge was found. Atrial lead. Its implantation at the free wall of the RAA near the origin of the RAA was verified. The lead was free of any adherent tissue or material. The tip of the electrode was visible from the epicardial surface through the epicardium, but no perforation was evident. Ventricular leads. Immediately distal to the CCTV venotomy site, the apical lead was enclosed in a tissue sheath 7 mm long. Distal to this, the lead was free of any adherence of tissue or other material until it reached its implant site in the RV apex. The other lead (RV wall), immediate distal to the CCTV venotomy site, had two pale nodules of adherent tissue or material, each <1 mm diameter and a very transparent 3 mm tissue sheath. Approximately 7 cm distal to the CCTV venotomy site, two segments of tissue sheaths, (5 mm and 2 mm long, respectively), were observed, neither sheath was adhered to adjacent cardiac tissues. This lead passed through the tricuspid valve at its caudal commissure. No adhesions were observed.

Dog Receiving Leads Coated With 5%DEX/PU.

Atrial lead. The lead that entered the venous system through the jugular vein had its distal end within the AVC (dislodged). A tissue sheath (1 cm long) was present around the lead body immediately distal to the jugular venotomy site. This lead was observed movable within its venotomy ligature. Within the RA, the septal wall, and around the RAA origin there were opaque thickenings of the endocardium. No other adherent tissue was observed on the rest of the body lead. Ventricular leads. Immediately distal to the CCTV venotomy site, a thin transparent 3 mm long tissue sheath, covering one lead was observed. The other lead had a more translucent tissue sheath 8 mm long, which was complicated distally by the presence of an eccentric, partly organized thrombus. This latter lead was adhered to the wall of the AVC. Distal to these tissue sheaths, both leads were free of any adherent tissue or material within the AVC or RA. Both leads passed through the tricuspid apparatus and remained free of any adhesions. A lead that passed more anteriorly in the RV was not implanted at its distal end but was freely movable. The lead that passed more caudal in the RVA was securely implanted and had a 3 mm sheath at its implantation site.

5. Histology of Intracardiac Lead-Associated Tissues

In general, occasional tissue sheaths were observed during the macroscopic pathologic evaluation. Lead-associated tissues, located at the lead portion from at least 1 cm distal from the venotomy site to at least 1 cm proximal to the electrode fixation site, were microscopically evaluated by a pathologist. One tissue sheath per condition was processed for histology, and a transversal section was stained with hematoxilin and eosin. The pathologist was kept blind to the treatment condition related to each specimen.

Although the results from the evaluation of these three specimens cannot be conclusive (n=1), they illustrate important findings that may be relevant to the different surface treatments in the study. A brief summary of the inflammatory characteristics and ranking of these slides follows. The least inflamed tissue is first:

Specimen From Dog Receiving Leads Coated With 5%DEX/PU.

Thinnest tissue sheath, no inner zone of partly organized thrombus, scant inflammation comprised of macrophages and few neutrophils.

Specimen From Dog Receiving Leads Coated With 1%DEX/PU.

Moderately thick tissue sheath, inner zone of partly organized thrombus, slightly more inflammation comprised of macrophages.

Specimen From Dog Receiving Leads Without DEX.

Moderately thick tissue sheath, inner zone of partly organized thrombus, moderate inflammation comprised of lymphocytes, plasma cells, macrophages, eosinophils, and few neutrophils.

6. Organ Evaluation

The regulatory influence of circulating steroids during the in vivo stage of implantation was evaluated. The negative feedback control of steroids on the neuroendocrine axis and on the anterior pituitary is well accepted. Prolonged suppression of ACTH release by steroids is associated with degenerative changes in the hypothalamus and in the anterior pituitary. These processes result in histopathological changes in the adrenal glands.

For these analyses, tissue sections of the adrenal glands were harvested from each animal. Other tissues for evaluation included liver, spleen, kidney, and lungs. Gross observations of the organs were documented by the pathologist. These tissue specimens were processed for routine histology studies.

In summary, the results showed no gross abnormalities in the evaluated organs. In the adrenal cortices of all dogs, the zona fasciculata cells had the foamy to vacuolated cytoplasm typical of active, steroid-secreting cells. There was no evidence microscopically of any lymphopenia, which might result from excessive administration of steroids. Microscopic evaluation of other organs revealed tissues with only minor abnormalities, none of which were attributable to corticosteroid coating on lead bodies. A consistent or progressive lymphopenia and/or eosinopenia was not noted in any dog in the study. Gross findings did include a) the presence of a body consistent with a gauze sponge found within the thoracic cavity of one of the dogs and b) bilateral subcutaneous carpal swelling of moderate size in another of the dogs. In general, all examined organs were found within normal limits.

7. DEX Elution Studies from Explanted Leads

The leads recovered immediately after pathological evaluation were subjected to in vitro elution in PBS at 37° C. The in vitro elution experiments are described above (“Kinetics of in vitro DEX Elution from DEX-Coated Pacing Leads”); analyses were conducted on eluates at 1 and 5 days of elution, and the DEX elution was calculated in terms of percentage of the initial DEX loadings in each lead (FIG. 12). The accumulated 5-day DEX release (in PBS) from explanted “low” and “high” lead conditions was 2.6% and 3.9% of the total DEX loading, respectively. This indicates that during the in vivo period of implantation, up to 90 days, DEX was still present for elution.

C. Conclusion

Coating, as a modality for applying the technology to devices, has been useful in the preparation of pacing lead prototypes and for demonstrating the feasibility of this concept. However, it is possible that extrusion and/or coextrusion of DEX/PU materials may be favorable for large-scale use and manufacturing of DEX-biomedical devices.

Overall, the in vivo study to evaluate the biological performance of DEX-pacing leads showed no complications. Results showed no DEX-related systemic toxicity during the 3-month implantation, as evidenced by hematological parameters or by histology of target organs. At the intracardiac portion of the leads (DEX-treated portion), minimal or no associated tissue encapsulation was observed in DEX-coated and control conditions. The observed tissue encapsulation were characterized as a typical reaction to polyurethane, as assessed macroscopically.

Microscopic histology of the occasional (intracardiac) lead-associated tissue sheaths (1 per condition), showed various degrees of inflammation, the intensity of which was inversely related to the presence of and the dose of DEX on the test device surfaces. These differential inflammatory findings in lead-associated tissue sheaths may suggest an active down-modulation of the cell functionality at the interface attributable to a localized DEX release. No systemic nor histological evidence of infection was found in the canines implanted with DEX-coated devices (n=4) or with control devices (n=2).

After 3 months of in vivo implantation, explanted DEX-coated leads showed a detectable DEX elution, with an accumulated elution of 2.6% and 3.9% of the total DEX at 5 days from “low” and “high” DEX-treatment conditions, respectively. This indicates the presence of DEX in the polymeric matrix during the in vivo period, suggesting an active DEX elution to the cell-biomaterial interface. Information on DEX release from explanted leads suggested that a sustained DEX release was still present after 3 months of in vivo implantation.

The complete disclosures of the patents, patent applications, and publications listed herein are incorporated by reference, as if each were individually incorporated by reference. The above examples and disclosure are intended to be illustrative and not exhaustive. These examples and description will suggest many variations and alternatives to one of ordinary skill in this art. All these alternatives and variations are intended to be included within the scope of the attached claims. Those familiar with the art may recognize other equivalents to the specific embodiments described herein which equivalents are also intended to be encompassed by the claims attached hereto.

We claim:

1. A medical electrical lead comprising;
an elongated insulative lead body having a tissue-contacting surface, a proximal end, and a distal end;
an elongated conductor having a proximal end and a distal end, mounted within the insulative lead body; and
an electrode coupled to the distal end of the electrical conductor for making electrical contact with bodily tissue;
wherein the tissue-contacting surface of the insulative lead body comprises a polymer in intimate contact with a steroidal anti-inflammatory agent.

2. The medical electrical lead of claim 1 wherein the polymer is selected from the group of polyurethanes, silicones, polyamides, polyimides, polycarbonates, polyethers, polyesters, polyvinyl aromatics, polytetrafluoroethylenes, polyolefins, acrylic polymers or copolymers, vinyl halide polymers or copolymers, polyvinyl ethers, polyvinyl esters, polyvinyl ketones, polyvinylidine halides, polyacrylonitriles, copolymers of vinyl monomers with each other and olefins, and combinations thereof.

3. The medical electrical lead of claim 2 wherein the polymer is selected from the group of polyurethanes, silicones, or combinations thereof.

4. The medical electrical lead of claim 1 wherein the anti-inflammatory agent is a glucocorticosteroid.

5. The medical electrical lead of claim 4 wherein the glucocorticosteroid is selected from the group of cortisol, cortisone, fludrocortisone, Prednisone, Prednisolone, 6α-methylprednisolone, triamcinolone, betamethasone, dexamethasone, beclomethasone, aclomethasone, amcinonide, clebethasol, clocortolone, derivatives thereof, and salts thereof.

6. The medical electrical lead of claim 5 wherein the glucocorticosteroid is dexamethasone, a derivative thereof, or a salt thereof.

7. The medical electrical lead of claim 1 wherein the anti-inflammatory agent is coated onto the tissue-contacting surface.

8. The medical electrical lead of claim 1 wherein the tissue-contacting surface comprises an anti-inflammatory agent incorporated into a polymeric overcoating.

9. The medical electrical lead of claim 1 wherein the anti-inflammatory agent is impregnated into the polymer of the tissue-contacting surface.

10. The medical electrical lead of claim 1 wherein the anti-inflammatory agent is covalently bonded to the polymer of the tissue-contacting surface.

11. The medical electrical lead of claim 1 wherein the tissue-contacting surface further includes heparin.

12. A medical electrical lead comprising:
an elongated insulative lead body having a tissue-contacting surface, a proximal end, and a distal end;
an elongated conductor having a proximal end and a distal end, mounted within the insulative lead body; and
an electrode coupled to the distal end of the electrical conductor for making electrical contact with bodily tissue;
wherein the tissue-contacting surface of the insulative lead body consists essentially of a nonporous polymer in intimate contact with a steroidal anti-inflammatory agent.

13. An indwelling catheter comprising:
an elongate body having a proximal end, a distal end, a tissue-contacting surface, and at least one interior lumen therethrough; and
an external fitting coupled to the proximal end;
wherein the tissue-contacting surface of the elongate body comprises a polymer in intimate contact with a steroidal anti-inflammatory agent.

14. The indwelling catheter of claim 13 further comprising one or more helical coils formed in the elongate body between the proximal and distal ends.

15. The indwelling catheter of claim 13 wherein the polymer is selected from the group of polyurethanes, silicones, polyamides, polyimides, polycarbonates, polyethers, polyesters, polyvinyl aromatics, polytetrafluoroethylenes, polyolefins, acrylic polymers or copolymers, vinyl halide polymers or copolymers, polyvinyl ethers, polyvinyl esters, polyvinyl ketones, polyvinylidine halides, polyacrylonitriles, copolymers of vinyl monomers with each other and olefins, and combinations thereof.

16. The indwelling catheter of claim 15 wherein the polymer is selected from the group of polyurethanes, silicones, or combinations thereof.

17. The indwelling catheter of claim 13 wherein the anti-inflammatory agent is a glucocorticosteroid.

18. The indwelling catheter of claim 17 wherein the glucocorticosteroid is selected from the group of cortisol, cortisone, fludrocortisone, Prednisone, Prednisolone, 6α-methylprednisolone, triamcinolone, betamethasone, dexamethasone, beclomethasone, aclomethasone, amcinonide, clebethasol, clocortolone, derivatives thereof, and salts thereof.

19. The indwelling catheter of claim 18 wherein the glucocorticosteroid is dexamethasone, a derivative thereof, or a salt thereof.

20. The indwelling catheter of claim 13 wherein the anti-inflammatory agent is coated onto the tissue-contacting surface.

21. The indwelling catheter of claim 13 wherein the tissue-contacting surface comprises an anti-inflammatory agent incorporated into a polymeric overcoating.

22. The indwelling catheter of claim 13 wherein the anti-inflammatory agent is impregnated into the polymer of the tissue-contacting surface.

23. The indwelling catheter of claim 13 wherein the anti-inflammatory agent is covalently bonded to the polymer of the tissue-contacting surface.

24. The indwelling catheter of claim 13 wherein the tissue-contacting surface further includes heparin.

25. An indwelling catheter comprising:
an elongate body having a proximal end, a distal end, a tissue-contacting surface, and at least one interior lumen therethrough; and
an external fitting coupled to the proximal end;
wherein the tissue-contacting surface of the elongate body consists essentially of a nonporous polymer in intimate contact with a steroidal anti-inflammatory agent.

26. A method of modulating tissue encapsulation of a medical electrical lead comprising implanting the lead into a patient, wherein the medical electrical lead comprises:
an elongated insulative lead body having a tissue-contacting surface, a proximal end, and a distal end;
an elongated conductor having a proximal end and a distal end, mounted within the insulative lead body; and
an electrode coupled to the distal end of the electrical conductor for making electrical contact with bodily tissue;
wherein the tissue-contacting surface of the insulative lead body comprises a polymer in intimate contact with a steroidal anti-inflammatory agent.

27. A method of modulating tissue encapsulation of an indwelling catheter comprising implanting the indwelling catheter into a patient, wherein the indwelling catheter comprises:
an elongate body having a proximal end, a distal end, a tissue-contacting surface, and at least one interior lumen therethrough; and
an external fitting coupled to the proximal end;
wherein the tissue-contacting surface of the elongate body comprises a polymer in intimate contact with a steroidal anti-inflammatory agent.

28. A method of modulating degradation of a medical electrical lead comprising implanting the lead into a patient, wherein the medical electrical lead comprises:
an elongated insulative lead body having a tissue-contacting surface, a proximal end, and a distal end;
an elongated conductor having a proximal end and a distal end, mounted within the insulative lead body; and
an electrode coupled to the distal end of the electrical conductor for making electrical contact with bodily tissue;
wherein the tissue-contacting surface of the insulative lead body comprises a polymer in intimate contact with a steroidal anti-inflammatory agent.

29. A method of modulating degradation of an indwelling catheter comprising implanting the indwelling catheter into a patient, wherein the indwelling catheter comprises:
an elongate body having a proximal end, a distal end, a tissue-contacting surface, and at least one interior lumen therethrough; and
an external fitting coupled to the proximal end;
wherein the tissue-contacting surface of the elongate body comprises a polymer in intimate contact with a steroidal anti-inflammatory agent.

30. A method of making a medical electrical lead comprising:
providing an elongated insulative lead body having a tissue-contacting surface, a proximal end, and a distal end; wherein the tissue-contacting surface comprises a polymer in intimate contact with a steroidal anti-inflammatory agent;
providing an elongated conductor having a proximal end and a distal end;
mounting the elongated conductor within the insulative lead body; and
coupling an electrode to the distal end of the electrical conductor for making electrical contact with bodily tissue.

31. The method of claim 30 wherein the step of providing an elongated insulative lead body comprises blending a steroidal anti-inflammatory agent with a polymer and forming a tissue-contacting surface.

32. The method of claim 30 wherein the step of providing an elongated insulative lead body comprises coating a steroidal anti-inflammatory agent onto the tissue-contacting surface of the lead body.

33. A method of making an indwelling catheter comprising:
providing an elongate body having a proximal end, a distal end, a tissue-contacting surface, and at least one interior lumen therethrough; wherein the tissue-contacting surface comprises a polymer in intimate contact with a steroidal anti-inflammatory agent; and
coupling an external fitting to the proximal end of the elongate body.

34. The method of claim 33 wherein the step of providing an elongate body comprises blending a steroidal anti-inflammatory agent with a polymer and forming a tissue-contacting surface.

35. The method of claim 30 wherein the step of providing an elongate body comprises coating a steroidal anti-inflammatory agent onto the tissue-contacting surface of the elongate body.
